(19) United States
(12) Patent Application Publication
(10) Pub. No.: US 2026/0272601 A1
(43) Pub. Date: Sep. 17, 2026

(54) SYSTEM AND METHOD FOR AUGMENTED REALITY SURGICAL PLANNING AND GUIDANCE WITH DYNAMIC REGISTRATION, BONE RESECTION VISUALIZATION, AND GAP BALANCING

(71) Applicant: PROVISION SURGICAL INC., DOVER, DE (US)

(72) Inventors: ABDULLAH ADNAN BAHOUR, NASHVILLE, TN (US); EN-HUA WANG, CENTREVILLE, VA (US); KEVIN WOO PARK, GREAT FALLS, VA (US); VINCENT MICHAEL CLAYTON, NASHVILLE, TN (US); HENRY ROBERT BOUCHER, BALTIMORE, MD (US); ABDURHMAN ADNAN BAHOUR, NASHVILLE, TN (US)

(73) Assignee: PROVISION SURGICAL, DOVER, DE (US)

(21) Appl. No.: 19/430,227

(22) Filed: Dec. 22, 2025

Related U.S. Application Data
(63) Continuation-in-part of application No. 19/169,236, filed on Apr. 3, 2025, now Pat. No. 12,514,646.
(60) Provisional application No. 63/737,784, filed on Dec. 22, 2024.
(60) Provisional application No. 63/737,786, filed on Dec. 22, 2024.

(51) Int. Cl. A61B 90/00; A61B 34/10; A61B 34/20; G06T 15/08; G06T 19/00; G09B 9/00

(52) U.S. Cl. CPC A61B 90/37; A61B 34/10; A61B 34/20; G06T 15/08; G06T 19/006; G09B 9/00; A61B 2034/105; A61B 2034/107; A61B 2034/2074; A61B 2090/365; G06T 2210/41

(57) ABSTRACT

A system for augmented reality surgical navigation may comprise an augmented reality surgical interface configured to be worn by a user and comprising a head-mounted display device with integrated cameras and sensors. A bone resection visualization module may generate real-time three-dimensional displays of resected areas and removed anatomy during surgical procedures. A gap balancing module may calculate flexion and extension gaps and provide automatic adjustment recommendations based on patient-specific biomechanical parameters. Imaging sensors may be positioned to monitor a surgical field and track changes in bone geometry during resection procedures. A processor may integrate visualization data from the bone resection visualization module and gap balancing calculations from the gap balancing module for coordinated display via the augmented reality surgical interface. The bone resection visualization module may generate volumetric representations of removed bone tissue with precise spatial positioning relative to remaining anatomical structures.

### CROSS-REFERENCE TO RELATED APPLICATIONS

Under provisions of 35 U.S.C. § 119(e), this application claims the benefit of U.S. Provisional Application No. 63/737,784, filed Dec. 22, 2024, entitled “Navigational System for Bone Resections with Enhanced Visualization of Resected Areas and Removed Anatomy”; and U.S. Provisional Application No. 63/737,786, filed Dec. 22, 2024, entitled “Navigational System for Knee Gap Balancing with Automatic Adjustments in an Augmented Reality Setting”. This application is also a continuation-in-part of U.S. patent application Ser. No. 19/169,236, entitled “System and Method for Augmented Reality Surgical Assistance with Integrated Biomechanical Metrics,” which claims priority to U.S. Provisional Application Nos. 63/654,922, 63/654,917, and 63/573,503. The entire contents of each of the above applications are hereby incorporated by reference herein. It is intended that each of the referenced applications may be applicable to the concepts and embodiments disclosed herein, even if such concepts and embodiments are disclosed in the referenced applications with different limitations and configurations and described using different examples and terminology.

### FIELD

The disclosure relates to augmented reality surgical navigation systems. More particularly, the disclosure relates to systems and methods that provide real-time visualization of bone resections, removed anatomy, and automated gap balancing during orthopedic surgical procedures.

### BACKGROUND

Traditional surgical methods for bone resections and implant placement may rely on manual estimation and two-dimensional imaging. Conventional approaches may limit accuracy and spatial understanding during orthopedic procedures. Existing systems may fail to provide real-time feedback on the precise location and size of resected areas. Traditional methods may not adequately visualize removed anatomy relative to remaining bone structures. Furthermore conventional methods may fail to account for changes in anatomical positioning during patient movement.

Current knee gap balancing techniques may depend on manual assessments and estimations by surgeons. Flexion and extension gaps may require precise equalization to ensure proper joint function and implant longevity. Conventional systems may lack real-time visualization of gap metrics during surgery. Furthermore, conventional systems may not dynamically account for changes in joint spacing resulting from patient motion, soft tissue behavior, or intraoperative repositioning. Manual resection adjustments may be imprecise and time-consuming.

Existing surgical navigation systems may not integrate comprehensive visualization of resected bone areas with automated gap balancing capabilities. There may be a need for systems that combine real-time augmented reality visualization with automated calculations and adjustments for improved surgical precision.

### SUMMARY

A system and method for augmented reality surgical navigation with integrated bone resection visualization and gap balancing may be provided. This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

In some embodiments, a system for augmented reality surgical navigation may comprise an augmented reality surgical interface configured to be worn by a user and a bone resection visualization module configured to generate real-time displays of resected areas and removed anatomy. The system may include a gap balancing module configured to calculate flexion and extension gaps and provide automatic adjustment recommendations. A processor may be configured to integrate the visualization and gap balancing data for display via the augmented reality surgical interface. The system may further include a registration module configured to align virtual anatomical models with physical anatomy using one or more registration modalities.

The system may provide real-time visualization of resected bone areas and volume of bone removed during surgery. The system may display interactive views of removed bone fragments and surrounding anatomy. The gap balancing module may calculate real-time gap metrics accounting for ligament tension, joint alignment, and patient-specific anatomy. The system may provide automatic or surgeon-guided adjustments to resection depths and angles based on live gap measurements. In one or more embodiments, the registration module may maintain alignment between virtual and physical anatomy during these adjustments.

In some embodiments, the augmented reality interface may overlay three-dimensional models and resection guidance onto the surgical field. The system may enable dynamic updates to display real-time visualization of resected areas, quantitative data on bone removal, and gap balancing metrics. The system may integrate with preoperative imaging data to provide continuous alignment and validation of surgical progress. In one or more embodiments, the system may support dynamic registration that updates alignment in response to patient movement or intraoperative repositioning

The bone resection visualization module may generate three-dimensional representations of removed bone fragments with volumetric calculations and spatial positioning relative to remaining anatomy. The system may provide enhanced visibility controls allowing surgeons to toggle between views of original anatomy, resected areas, and planned resection boundaries. The visualization module may support real-time updates as resection procedures progress, maintaining accurate spatial relationships between virtual and physical elements.

The gap balancing module may incorporate patient-specific biomechanical parameters including ligament properties, joint kinematics, and anatomical variations. The system may calculate optimal resection adjustments to achieve target gap measurements in both flexion and extension positions. The module may provide predictive modeling of gap changes based on proposed resection modifications, enabling surgeons to evaluate multiple scenarios before making cuts.

In some embodiments, the system may include a surgical training module configured to provide immersive educational experiences using the integrated visualization and gap balancing capabilities. The training module may present standardized surgical scenarios with quantitative performance metrics and real-time feedback on technique variations. The system may support collaborative training environments where multiple users can interact with shared virtual anatomical models and surgical instruments. In one or more embodiments, registration may be performed using manual landmark acquisition via a tracked finger or tracked stylus, automated visual detection, or combinations thereof.

The system may incorporate comprehensive data capture and analysis capabilities for quality assurance and outcome correlation. The system may record detailed metrics on resection geometry, gap measurements, surgical timing, and deviation from planned parameters. The data capture module may enable post-operative analysis and correlation with patient outcomes for continuous improvement of surgical techniques and system algorithms.

In some embodiments, the system may provide integration with robotic surgical platforms and automated instrument positioning systems. The gap balancing calculations may be used to guide robotic resection tools with real-time adjustments based on measured parameters. The system may support both fully automated and surgeon-supervised modes of operation, allowing for varying levels of human oversight and intervention during procedures.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

### BRIEF DESCRIPTION OF THE DRAWINGS

The accompanying drawings, which are incorporated in and constitute a part of this specification, illustrate exemplary embodiments of the disclosure and, together with the description, serve to explain the principles of the disclosure.

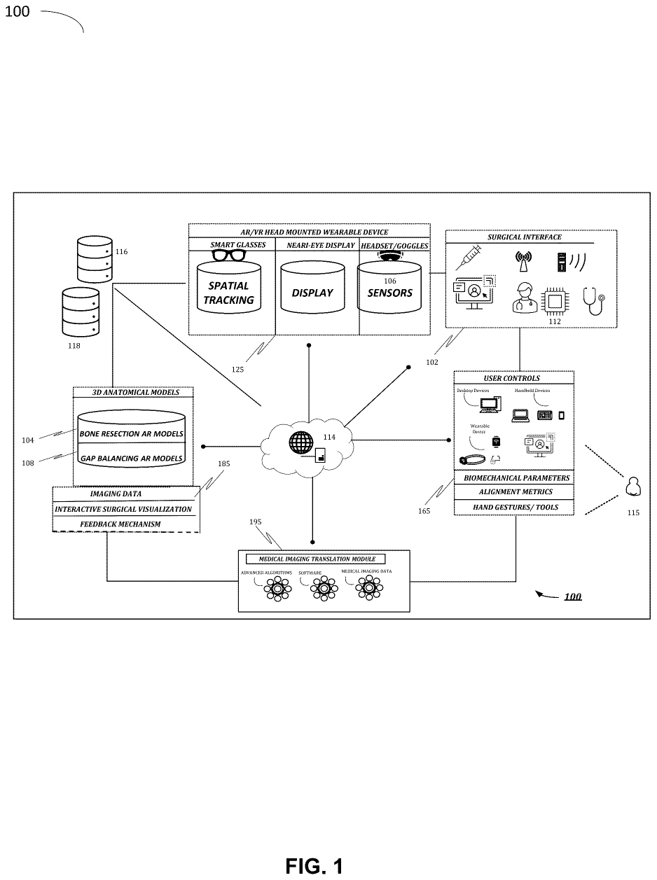

FIG. 1 is a schematic diagram illustrating a system architecture for the AR surgical assistance system, in accordance with embodiments of the present disclosure.

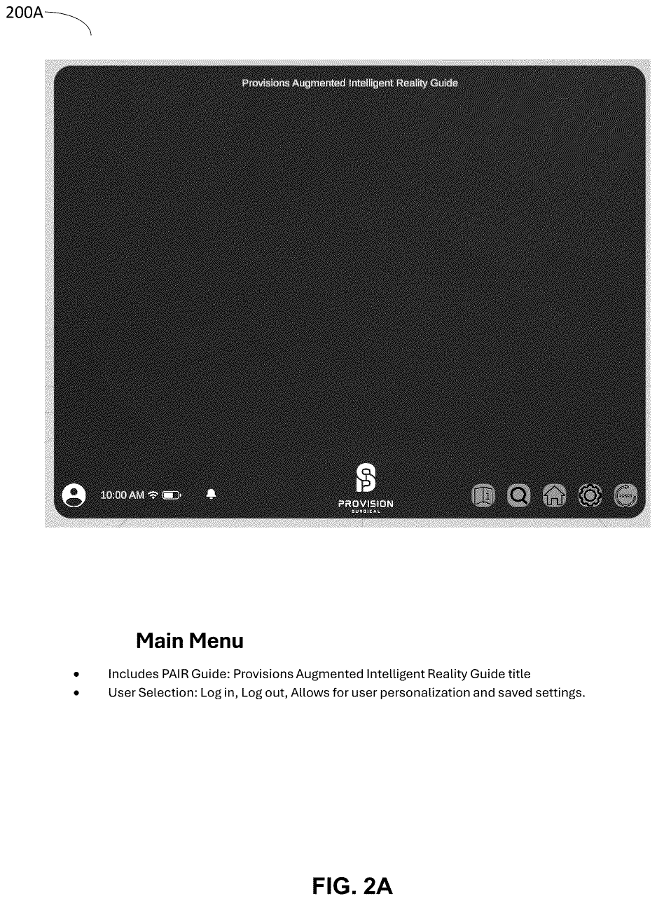

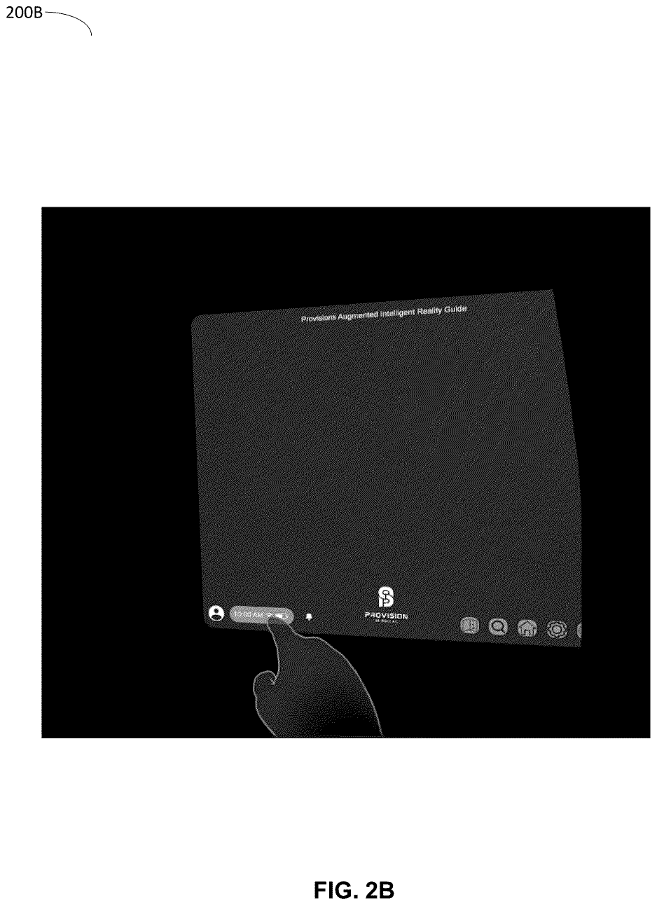

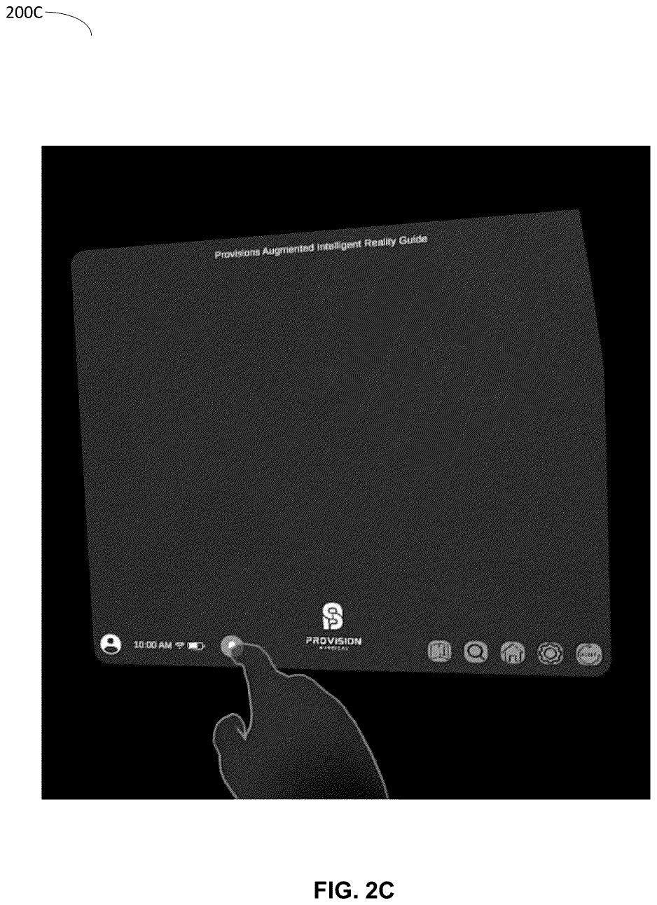

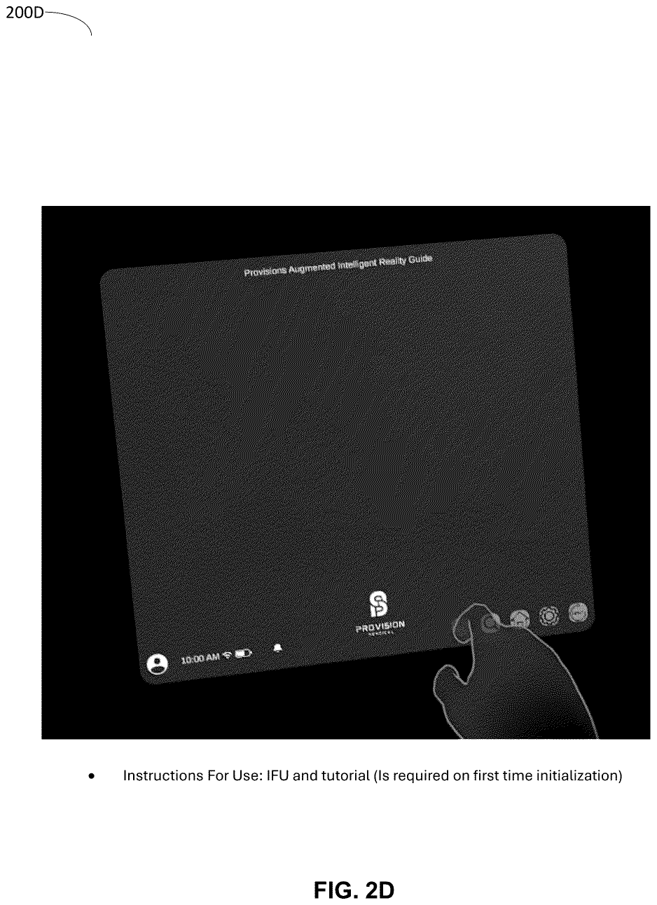

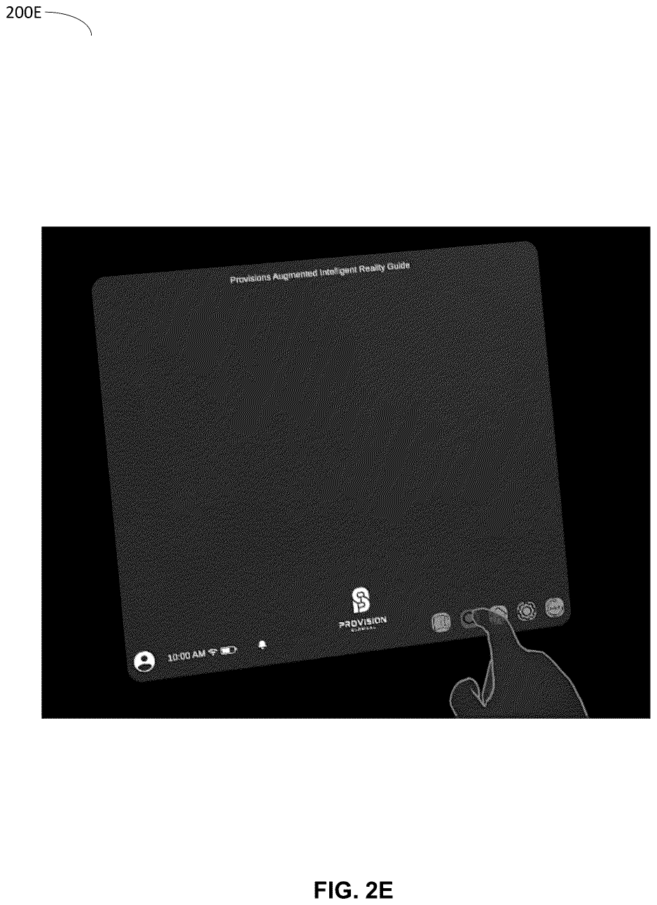

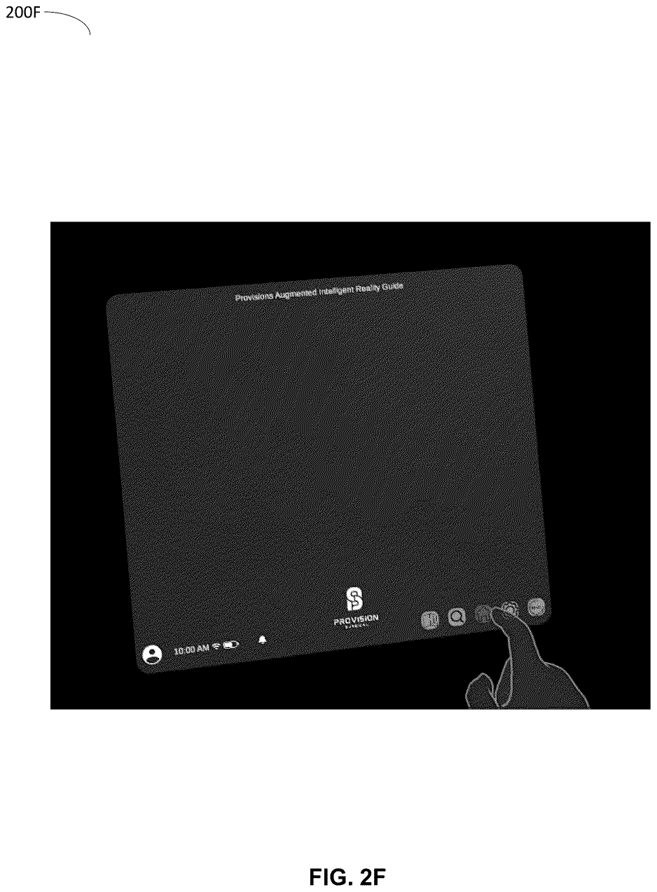

FIG. 2A, FIG. 2B, FIG. 2C, FIG. 2D, FIG. 2E, and FIG. 2F are schematic diagrams illustrating a Bone Alignment and Resection Planning Flow, Implant Sizing, Fit, and Positioning Flow, in accordance with embodiments of the present disclosure.

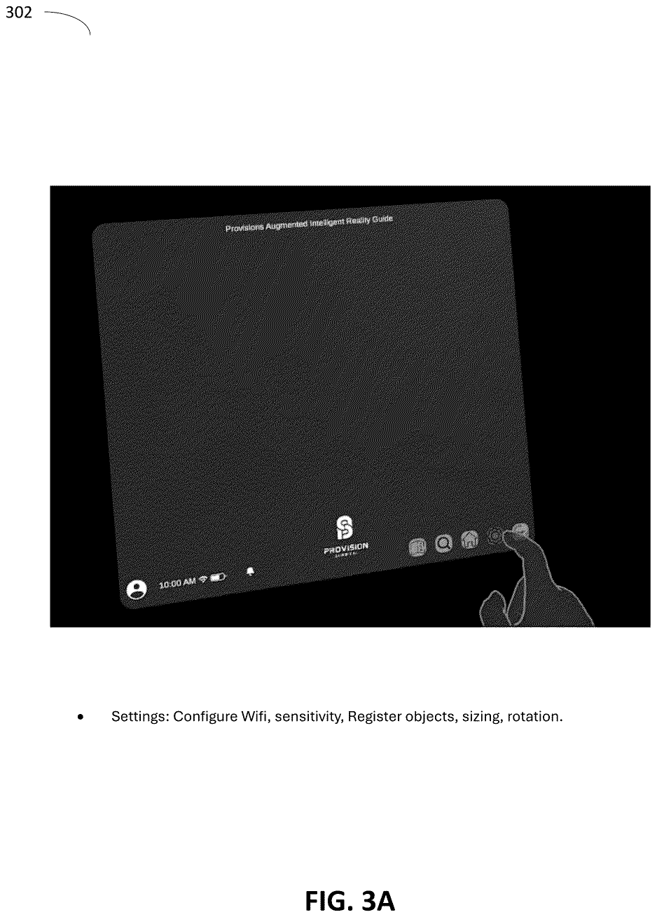

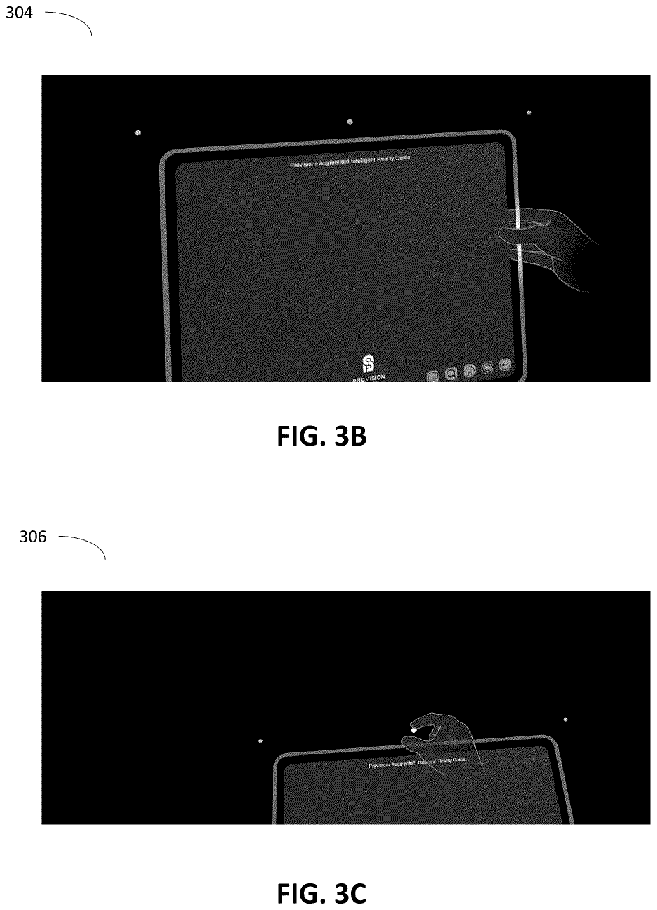

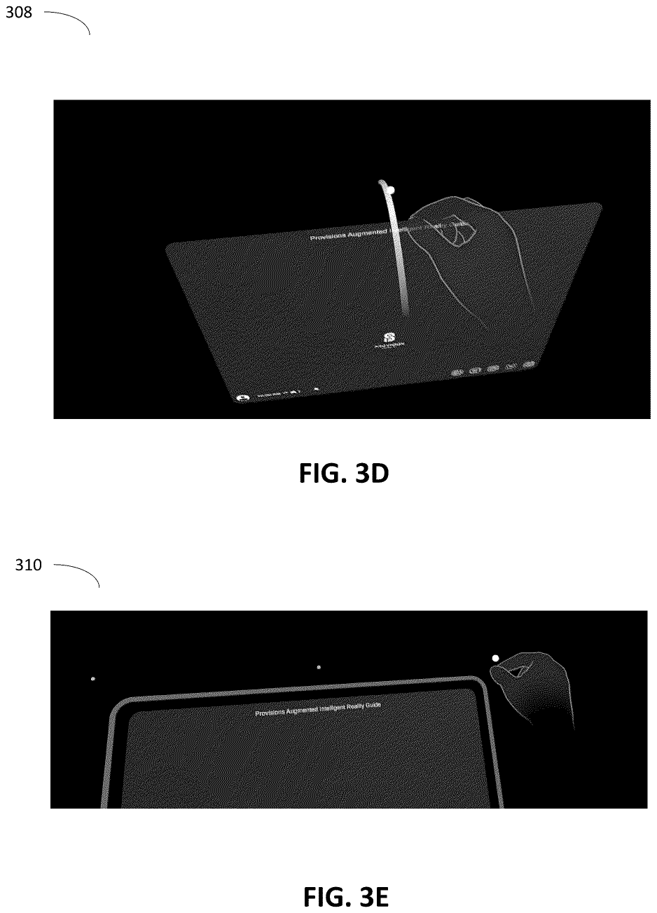

FIG. 3A, FIG. 3B, FIG. 3C, FIG. 3D, and FIG. 3E are schematic diagrams illustrating a Surgical Case Scheduling and Data Management Flow, in accordance with embodiments of the present disclosure.

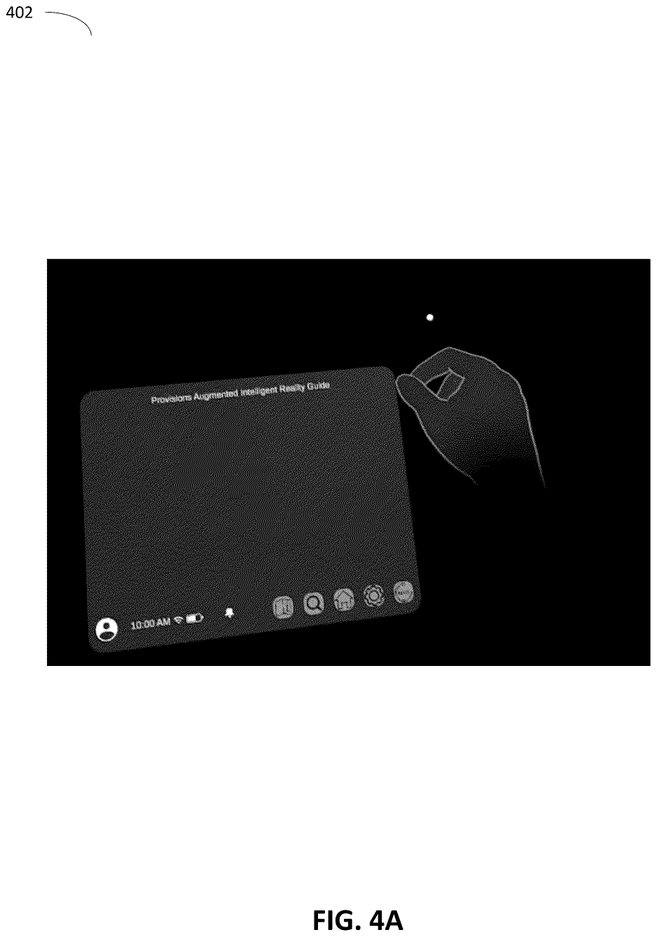

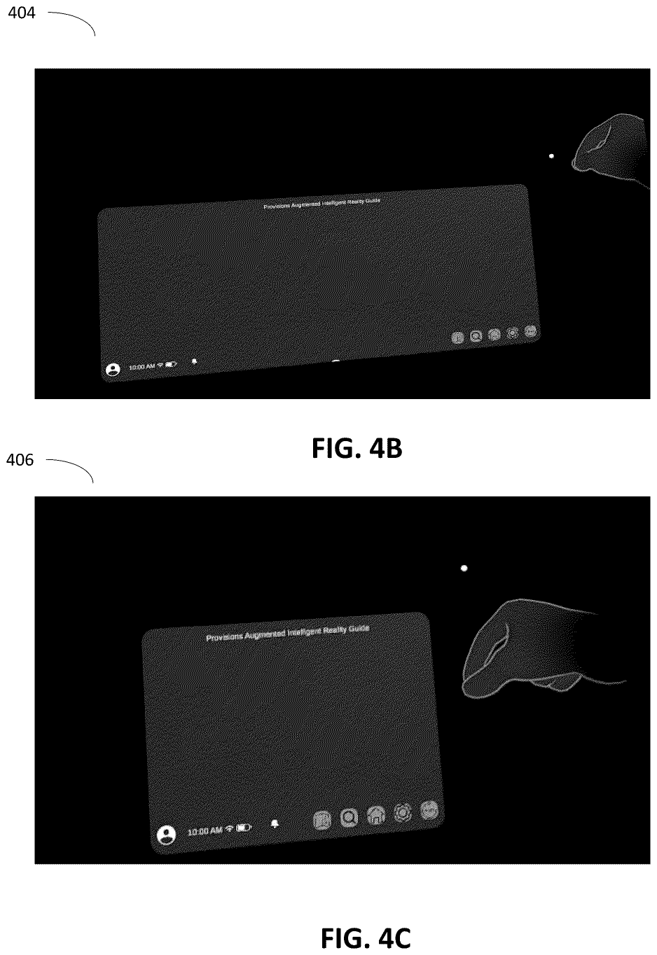

FIG. 4A, FIG. 4B, and FIG. 4C are schematic diagrams illustrating an Automated Processing & Surgical Planning Flow, in accordance with embodiments of the present disclosure.

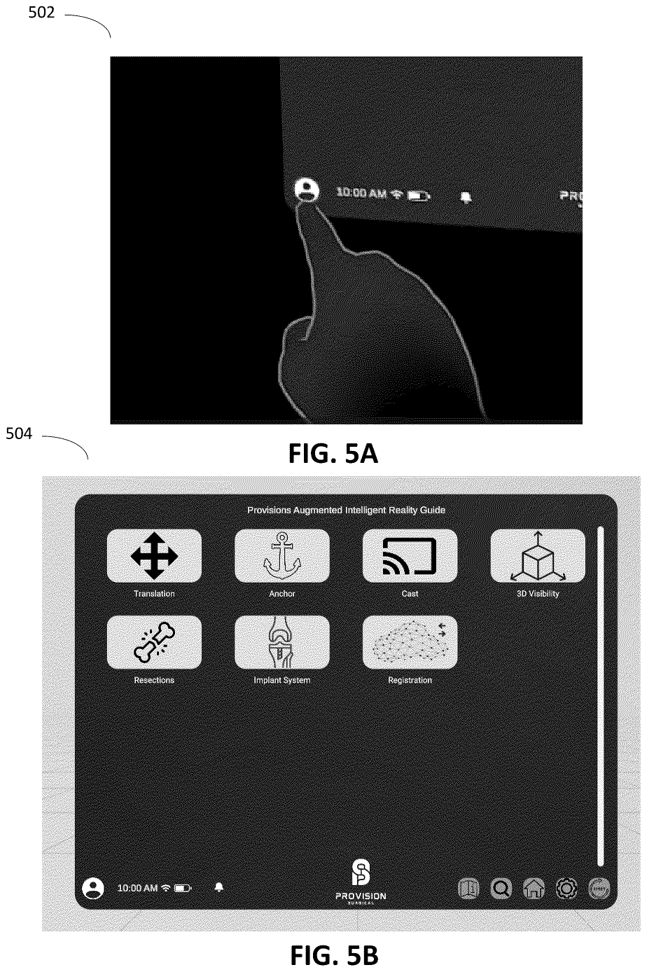

FIG. 5A and FIG. 5B are schematic diagrams illustrating a Loading Process in Unity App for Surgery Flow, in accordance with embodiments of the present disclosure.

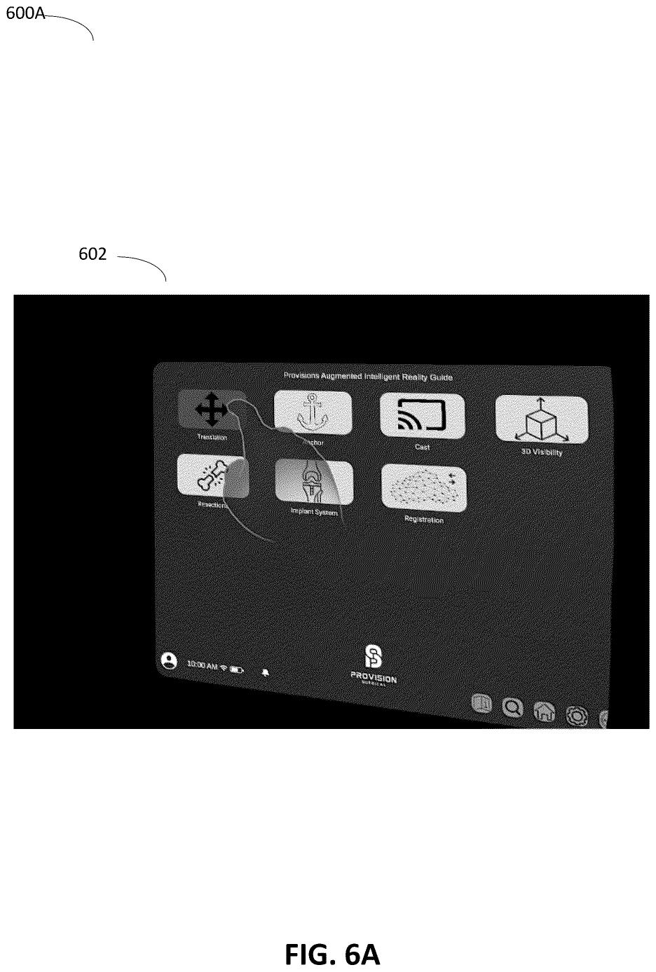

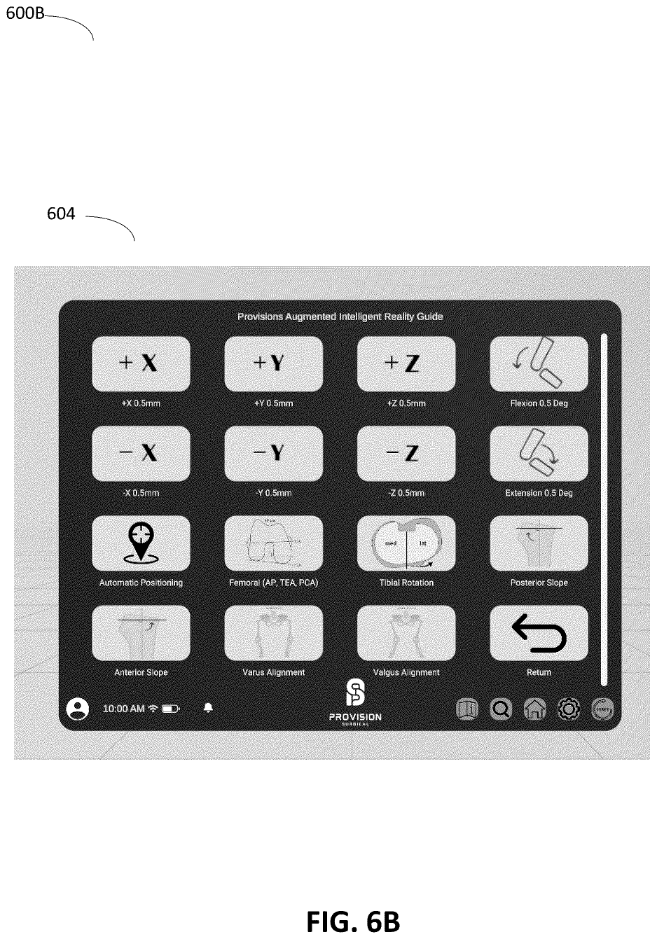

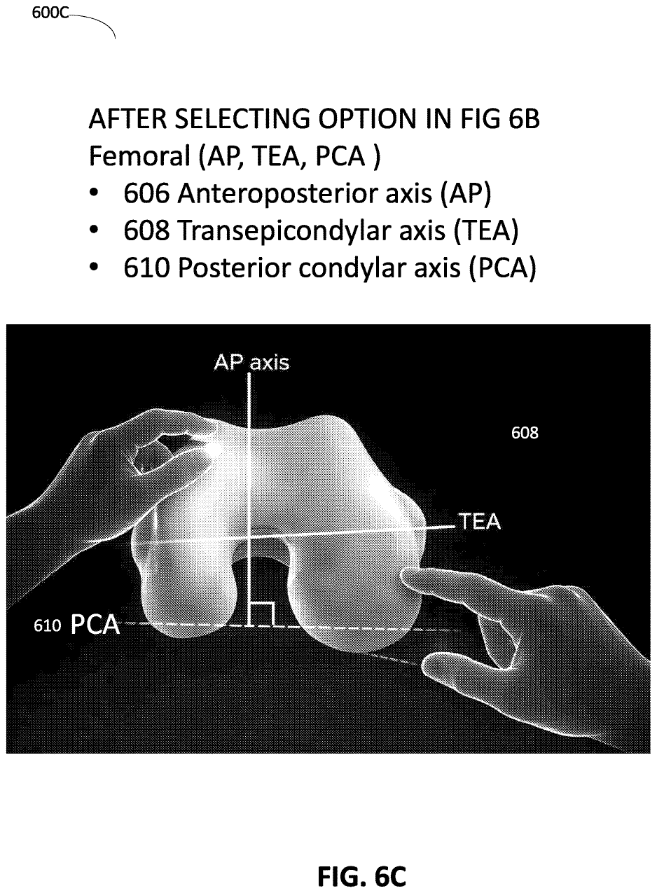

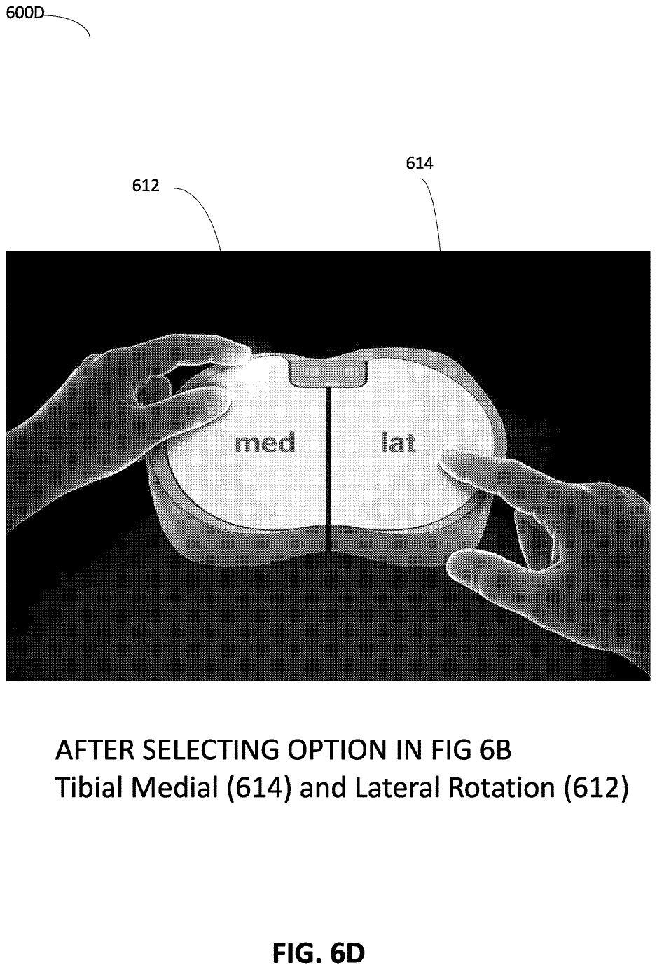

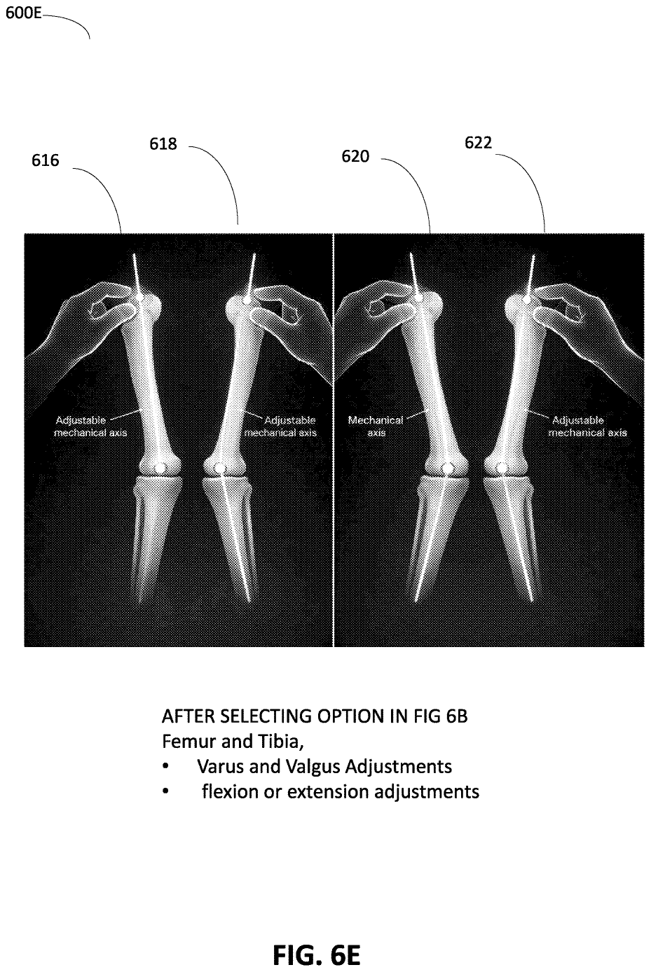

FIG. 6A, FIG. 6B, FIG. 6C, FIG. 6D, and FIG. 6E are schematic diagrams illustrating an Intraoperative AR Guidance Process Flow, in accordance with embodiments of the present disclosure.

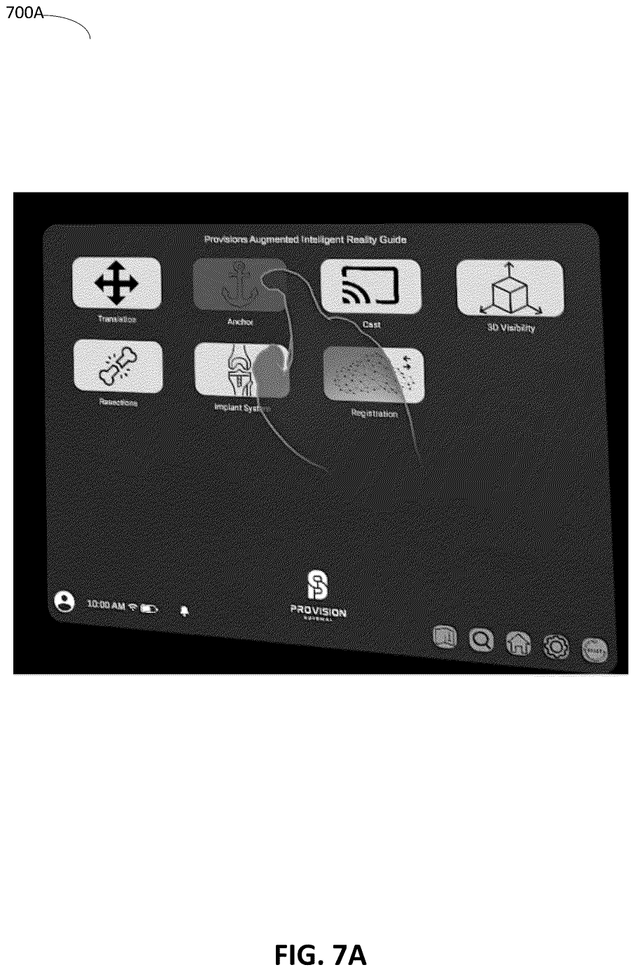

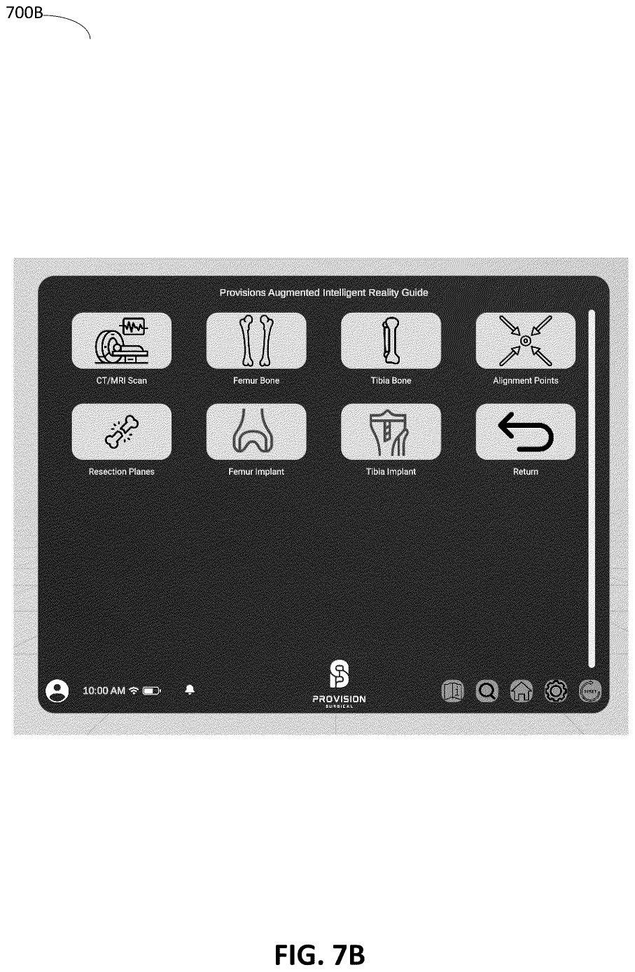

FIG. 7A and FIG. 7B are schematic diagrams illustrating a Postoperative Documentation Process Flow, in accordance with embodiments of the present disclosure.

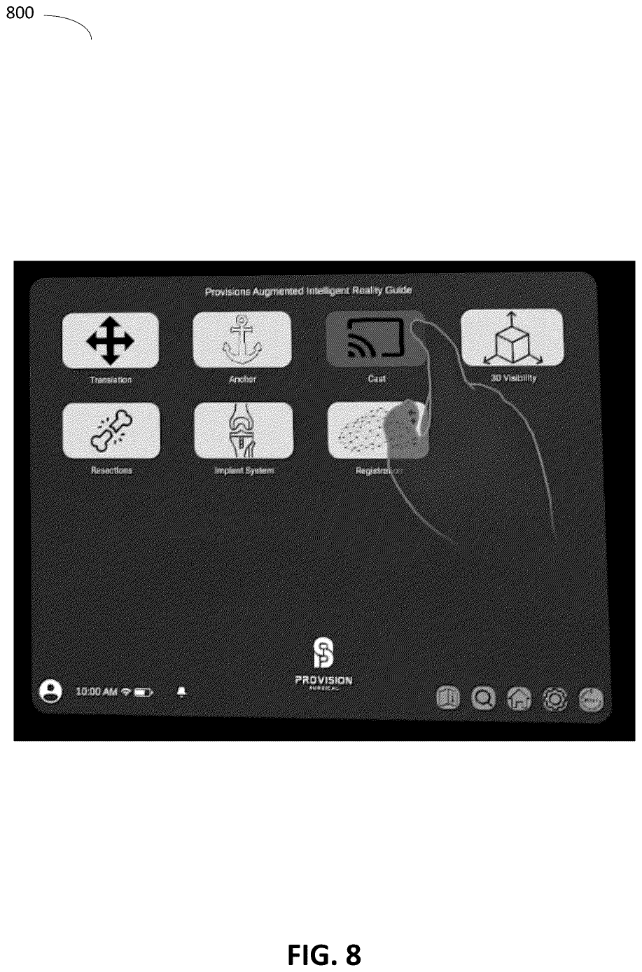

FIG. 8 is a schematic diagram illustrating a Surgical Case Scheduling and Data Management Flow, in accordance with embodiments of the present disclosure.

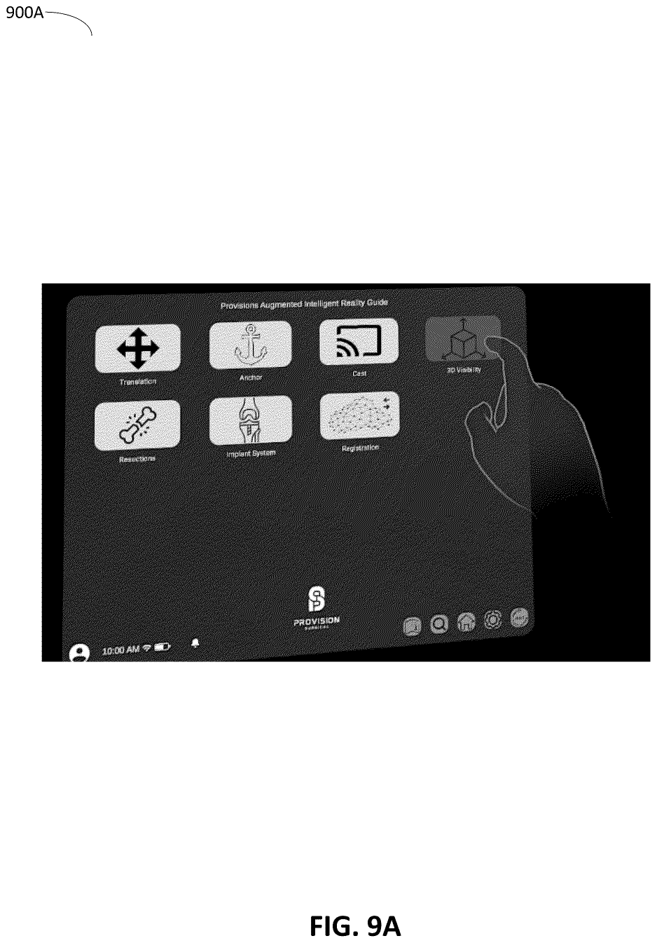

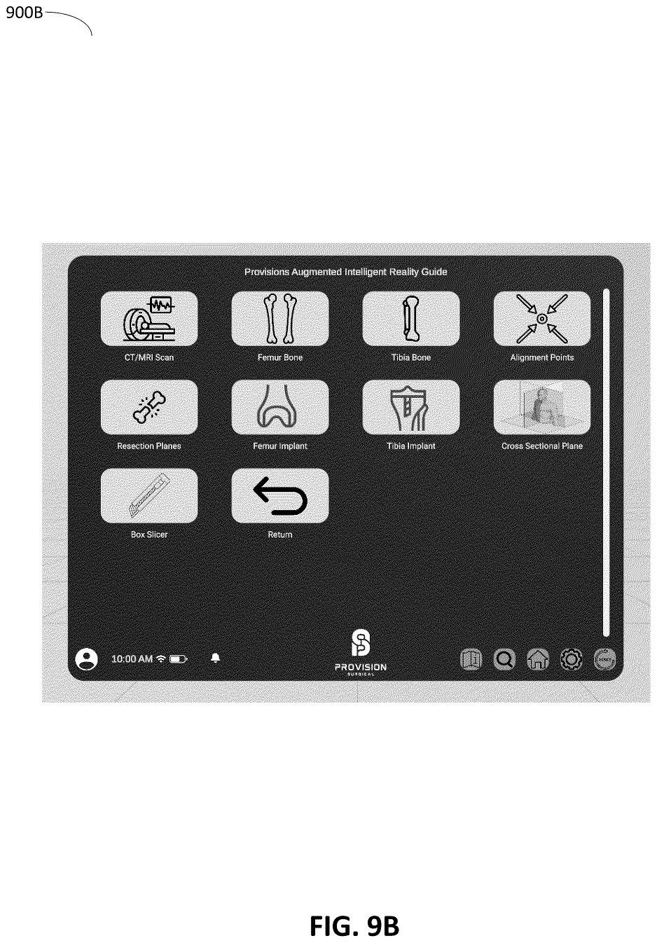

FIG. 9A and FIG. 9B are schematic diagrams illustrating an Automated Processing & Surgical Planning Flow, in accordance with embodiments of the present disclosure.

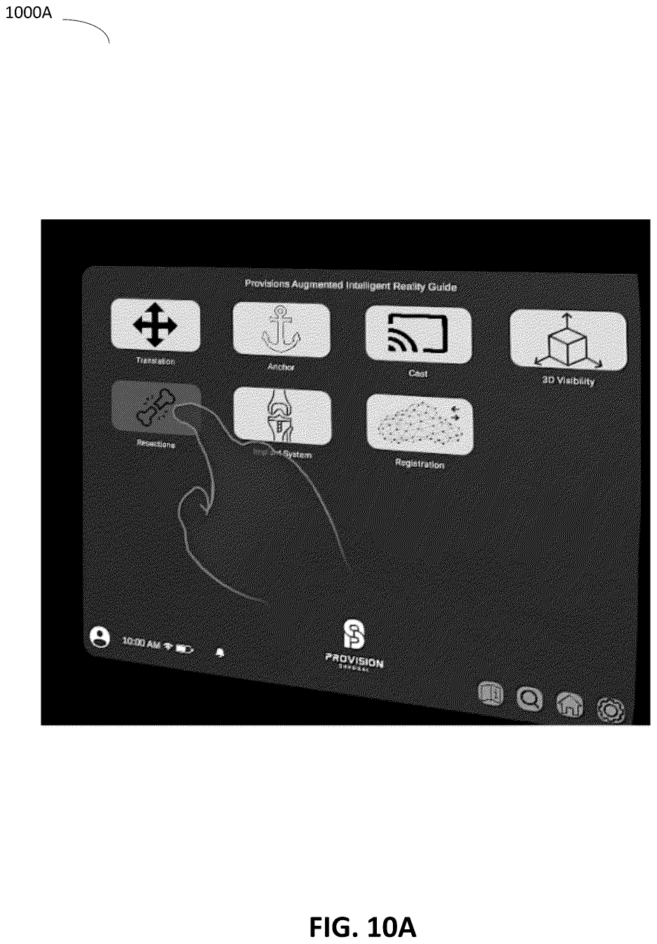

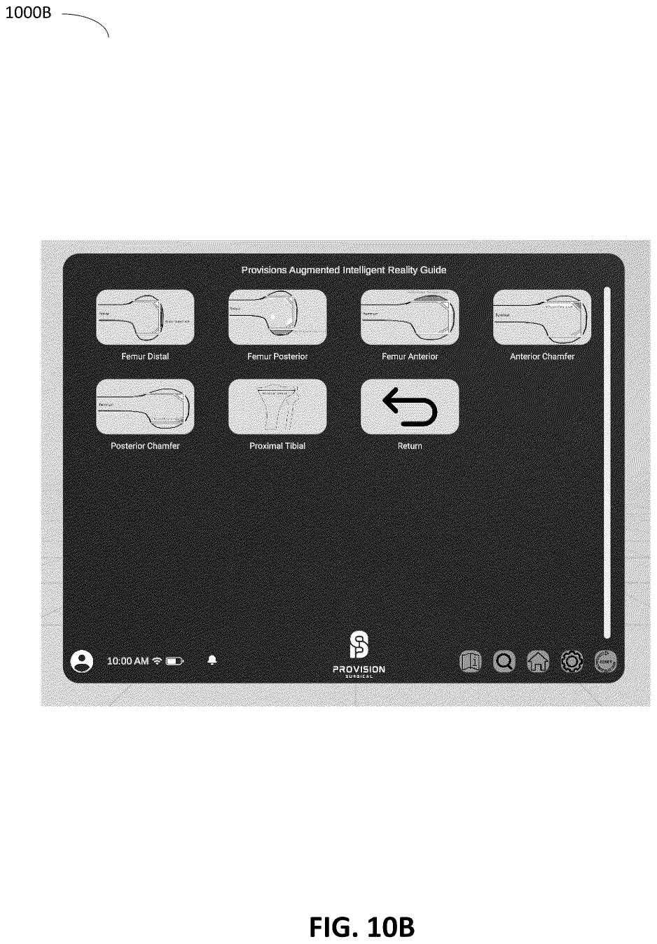

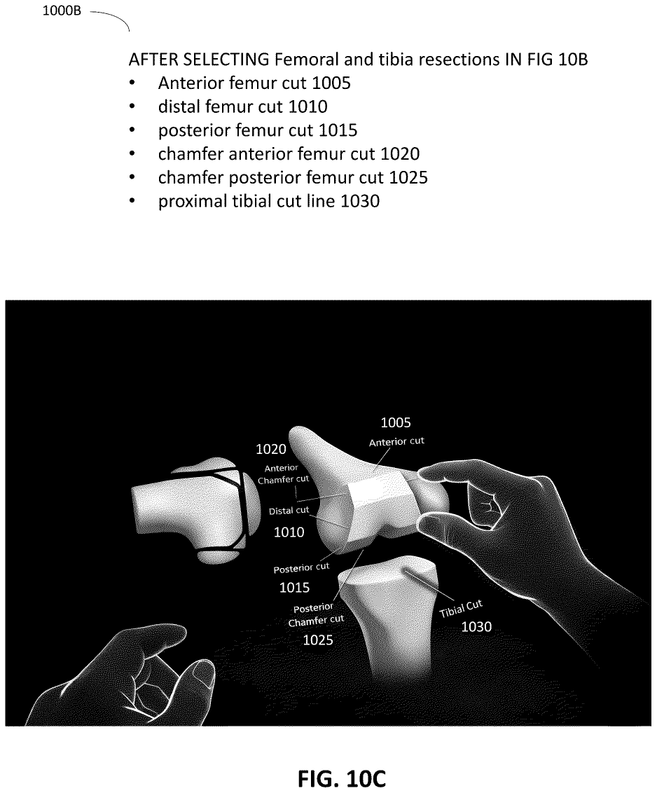

FIG. 10A, FIG. 10B, and FIG. 10C are schematic diagrams illustrating an Augmented Reality Surgical Guidance Flow, in accordance with embodiments of the present disclosure.

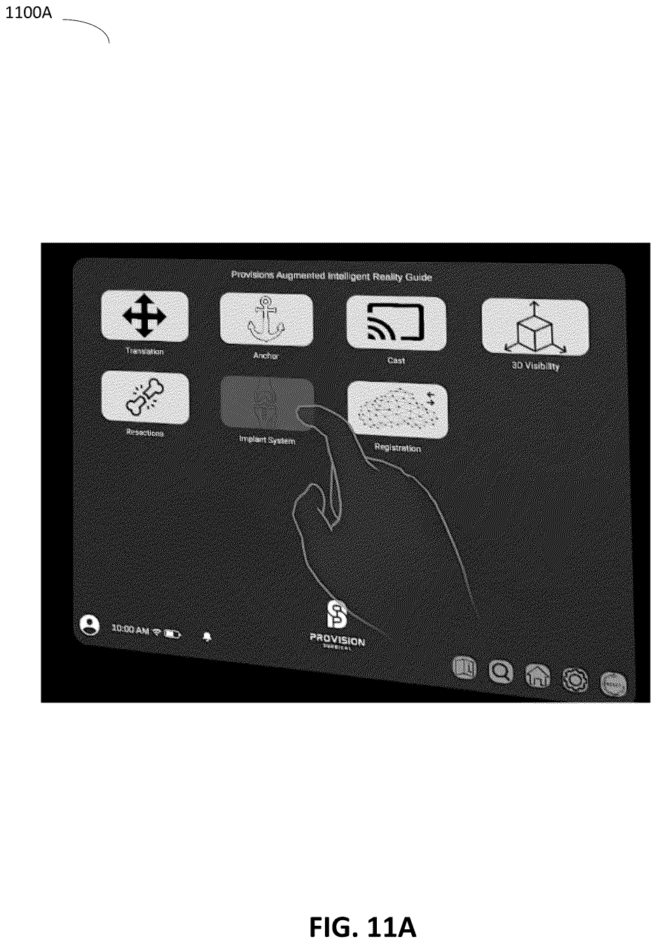

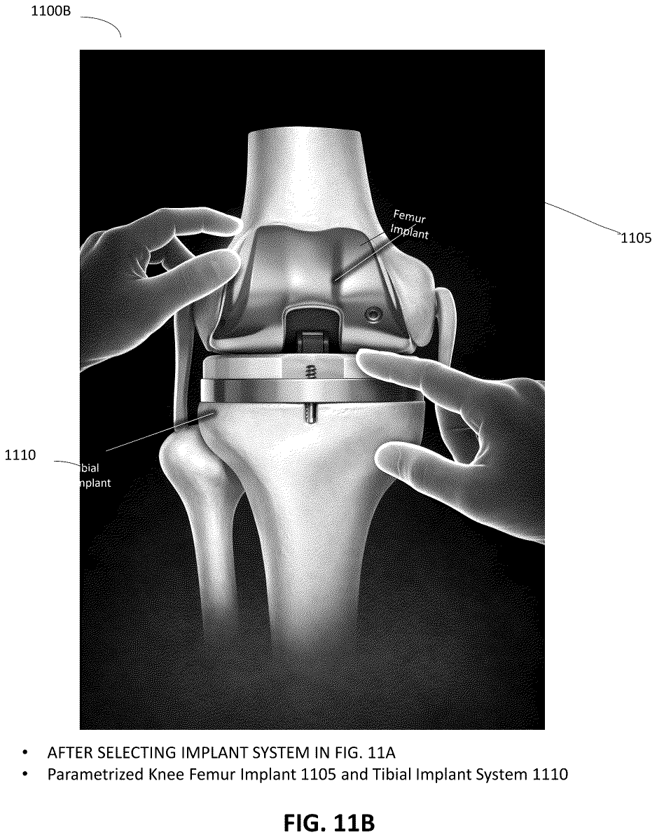

FIG. 11A and FIG. 11B are schematic diagrams illustrating an Intraoperative Guidance Process Flow, in accordance with embodiments of the present disclosure.

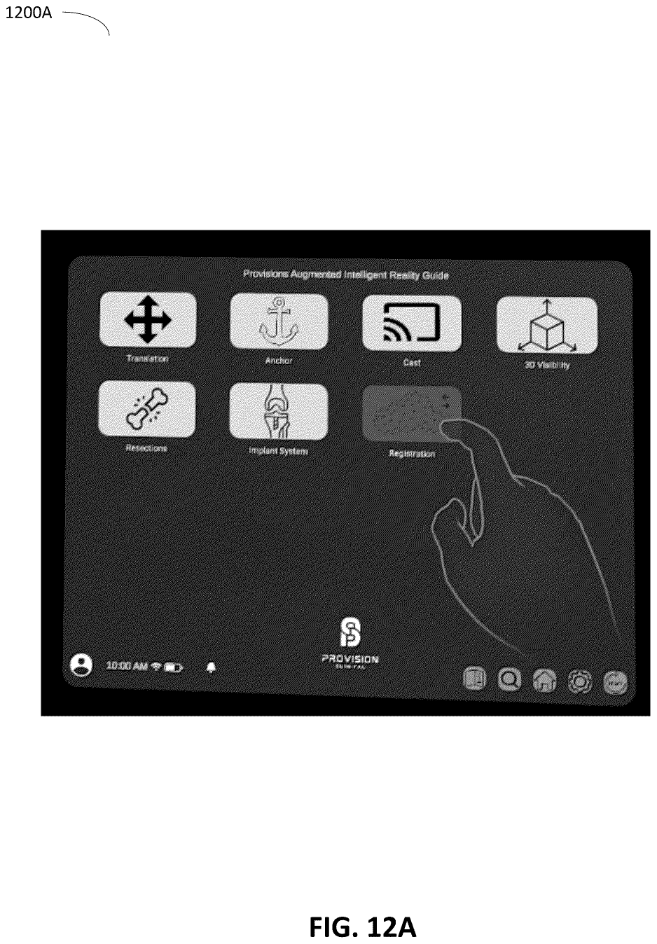

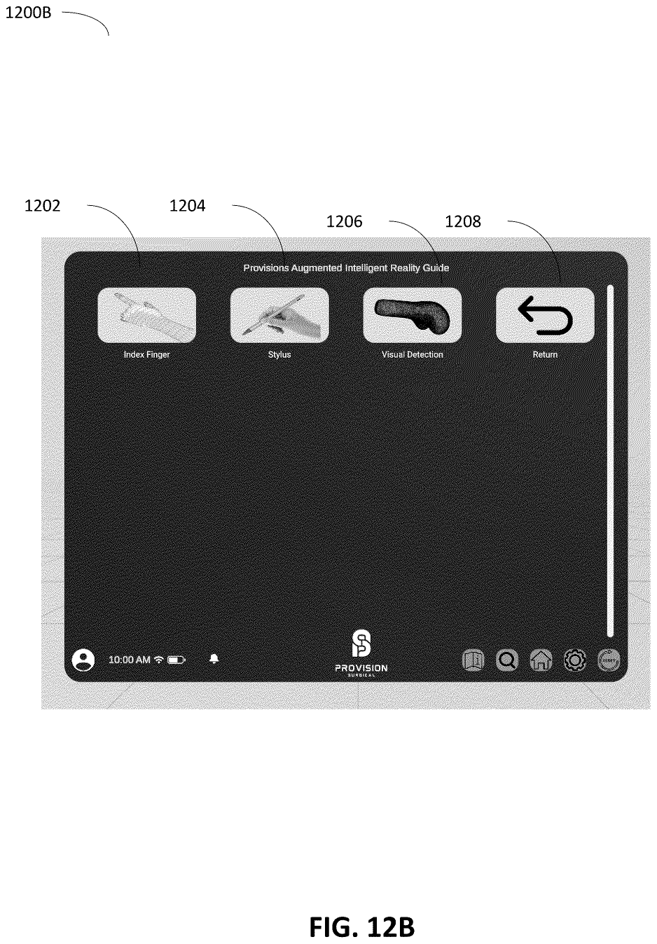

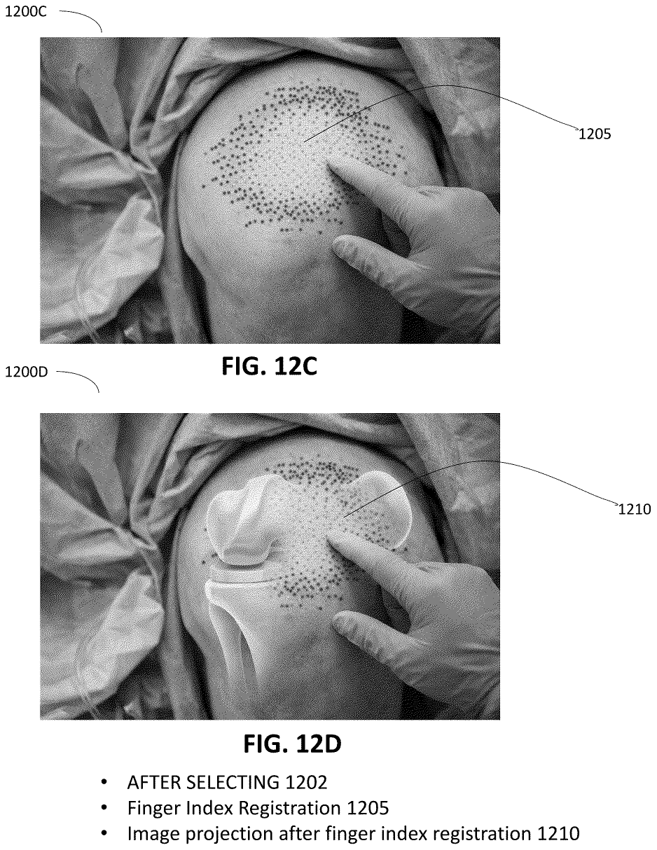

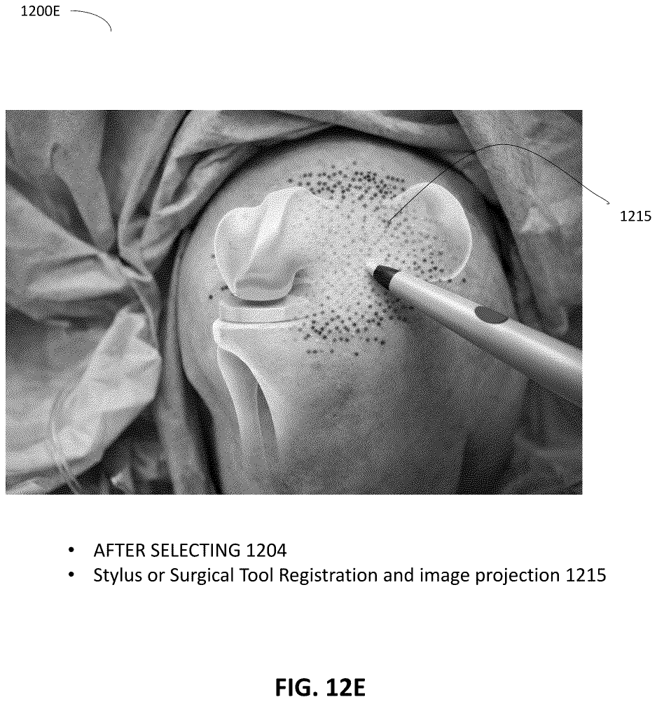

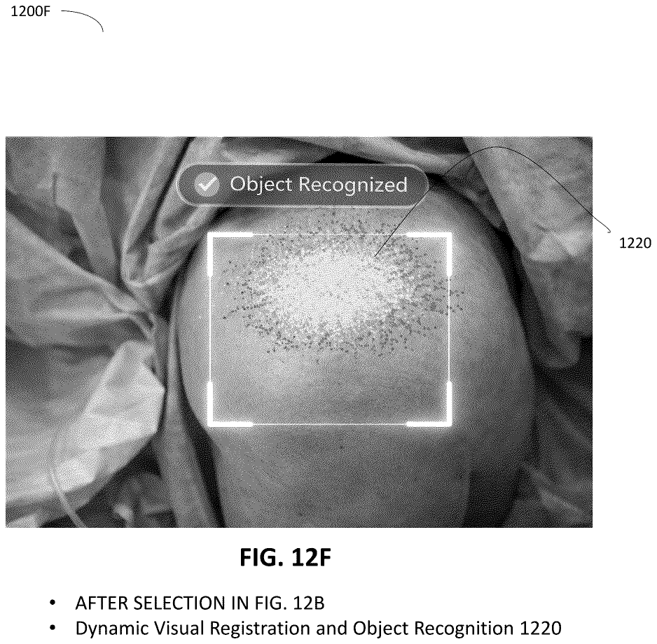

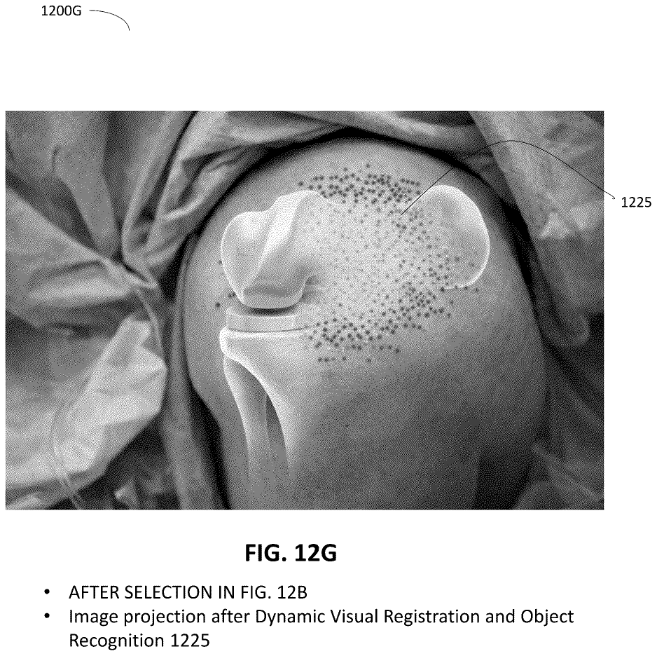

FIG. 12A, FIG. 12B, FIG. 12C, FIG. 12D, FIG. 12E, FIG. 12F, and FIG. 12G are schematic diagrams illustrating a Postoperative Documentation Process Flow, in accordance with embodiments of the present disclosure.

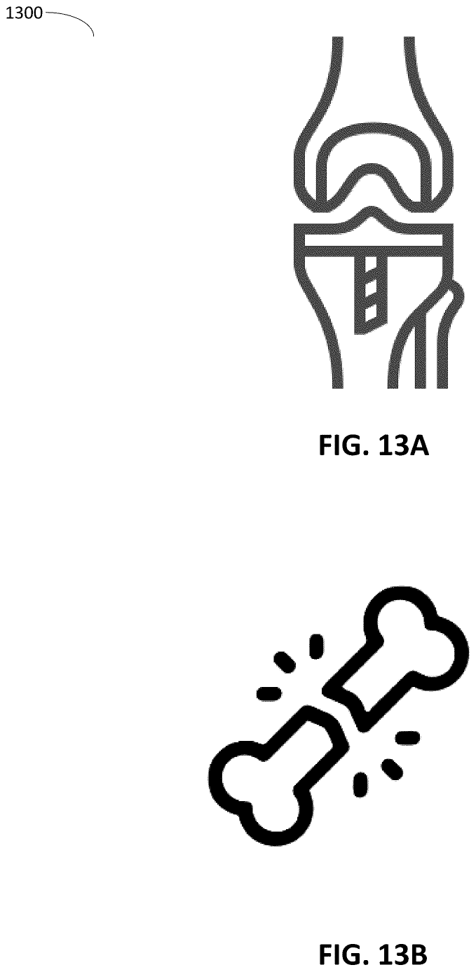

FIG. 13A and FIG. 13B are schematic diagrams illustrating a graphical user interface implemented in a display of a head mounted wearable device presenting a slicer tool box image, a slicer tool plane, a femur 3D model rendering, and a tibia 3D model rendering, in accordance with embodiments of the present disclosure.

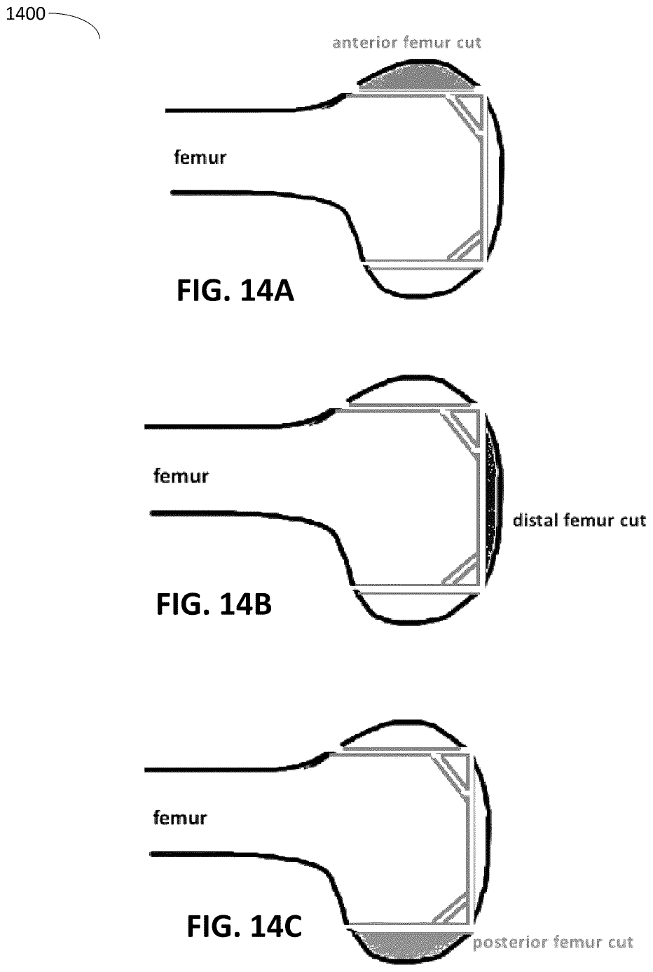

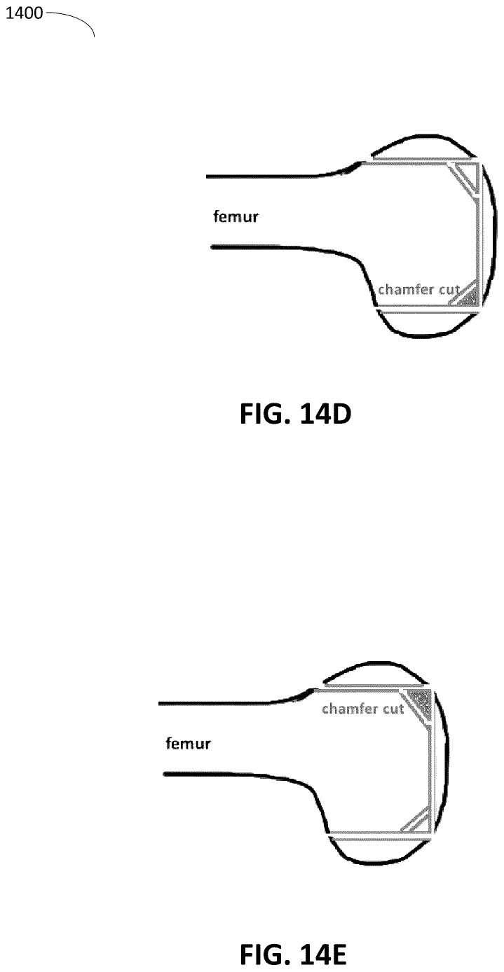

FIG. 14A, FIG. 14B, FIG. 14C, FIG. 14D, and FIG. 14E are schematic diagrams illustrating a GUI implemented in a display of a head mounted wearable device presenting a visibility menu image for anterior, distal, and posterior femur cuts, in accordance with embodiments of the present disclosure.

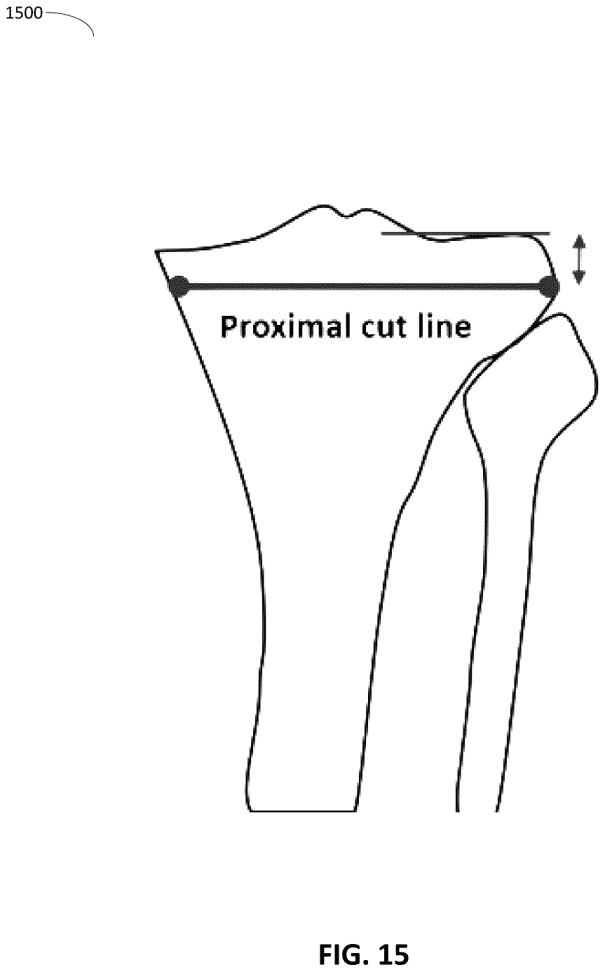

FIG. 15 is a schematic diagram illustrating a GUI implemented in a display of a head mounted wearable device presenting proximal cut lines, in accordance with embodiments of the present disclosure.

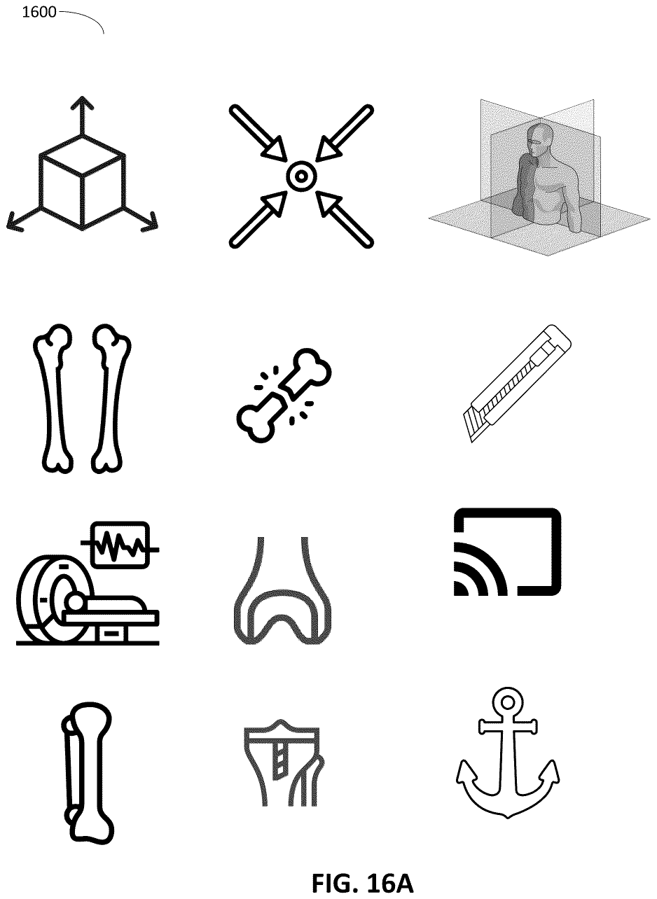

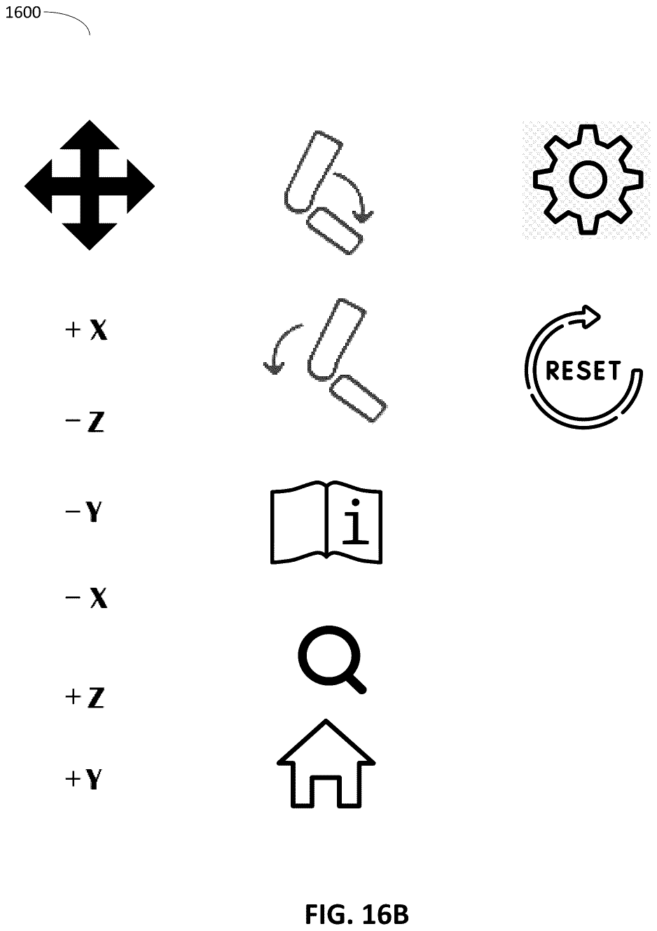

FIG. 16A and FIG. 16B are schematic diagrams illustrating the icons for selecting various elements for the head mounted wearable device allowing for AR assistance surgical guidance, in accordance with embodiments of the present disclosure.

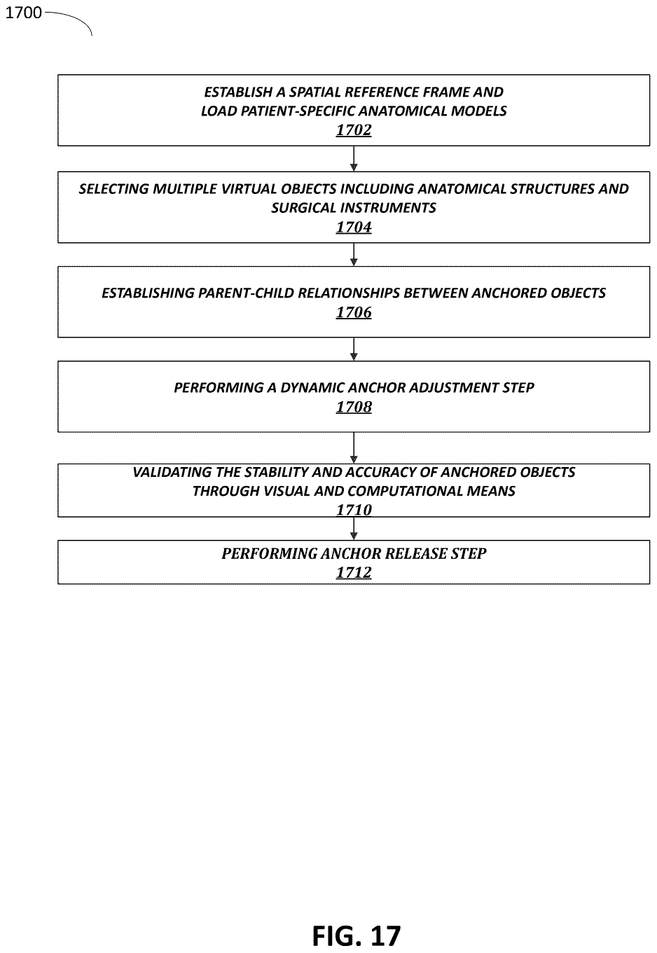

FIG. 17 is a method of operating a virtual locking system for three-dimensional visualized models

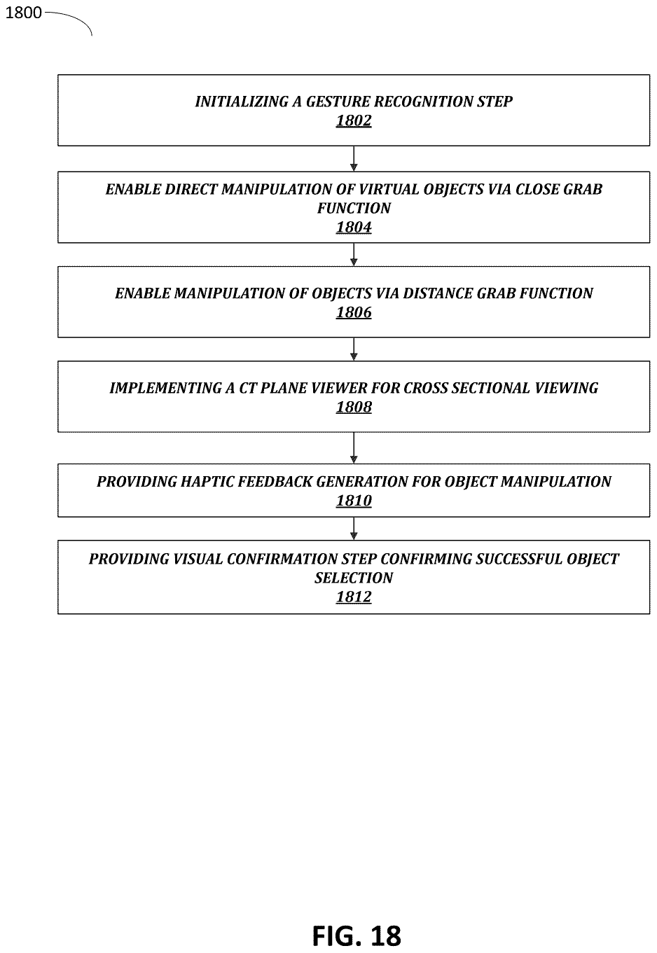

FIG. 18 is a method of providing hand interaction functionality in an augmented reality surgical environment.

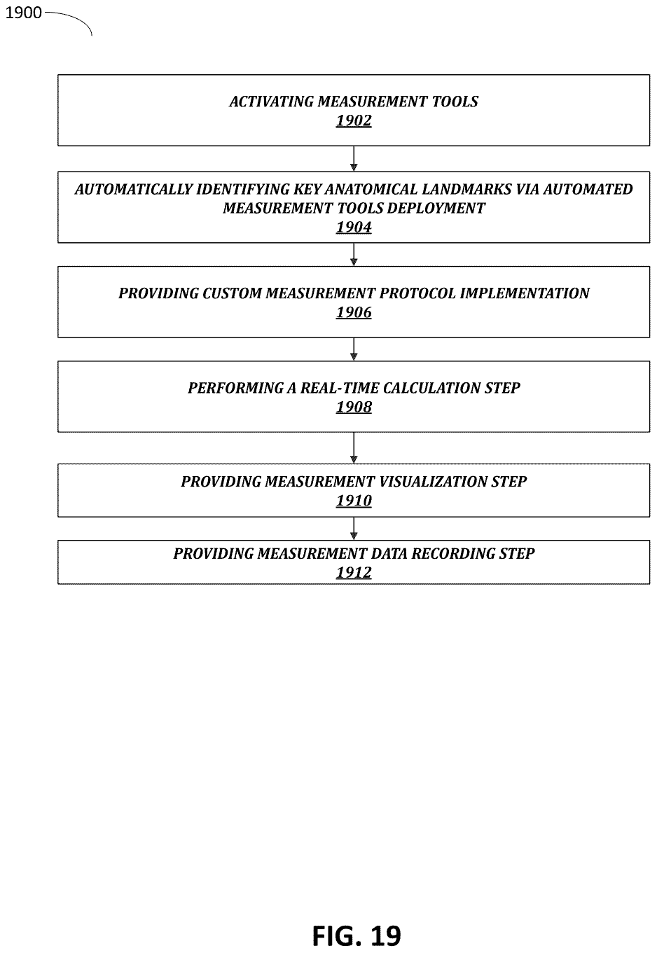

FIG. 19 is a method of providing measurement functionality in an augmented reality surgical environment.

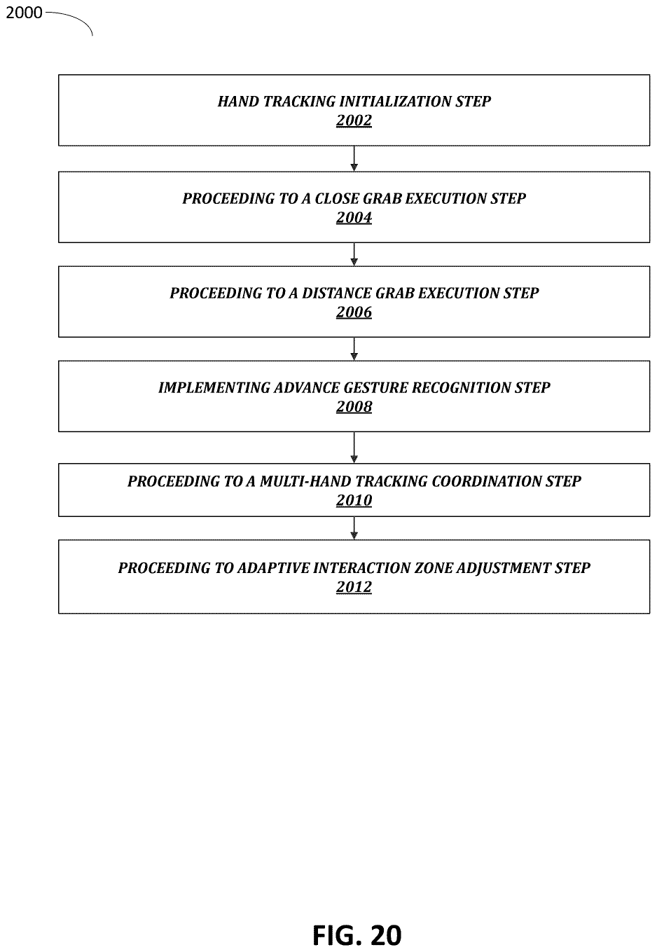

FIG. 20 is a method of providing hand interaction functionality in an augmented reality surgical environment.

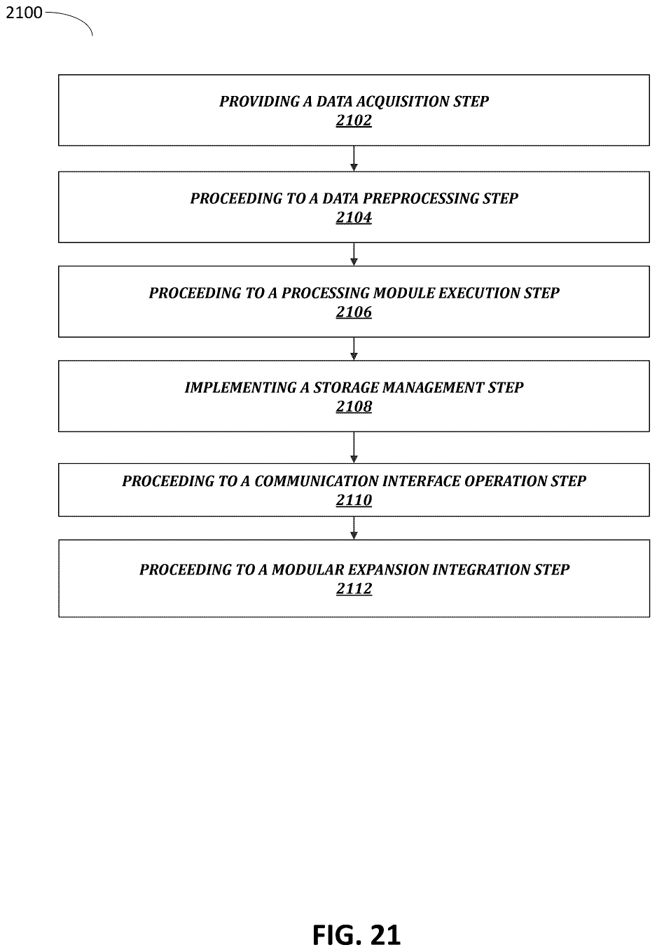

FIG. 21 is a flowchart showing the system for augmented reality surgical navigation.

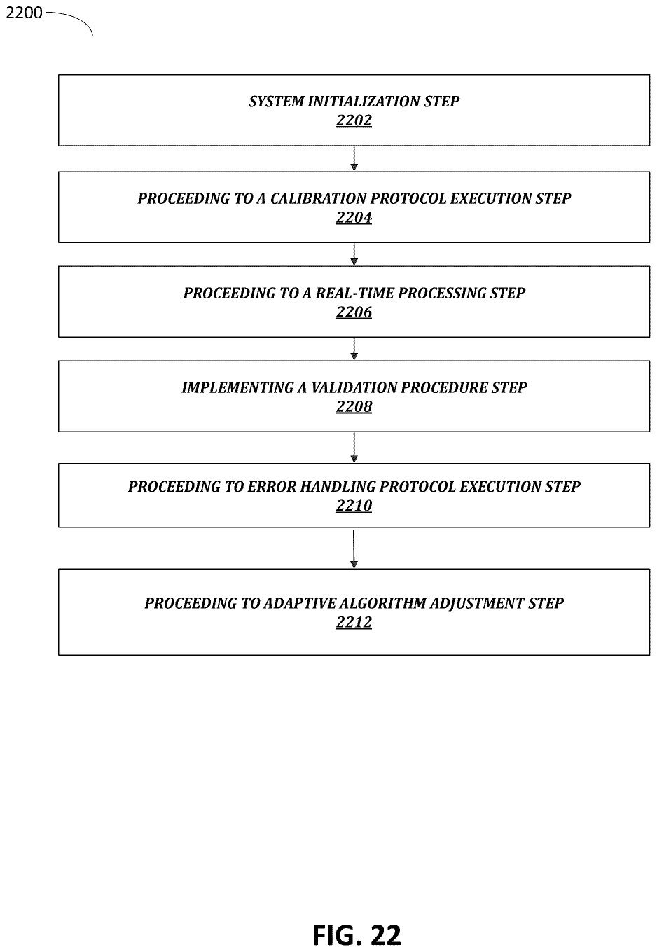

FIG. 22 is a flowchart showing the system for augmented reality surgical navigation.

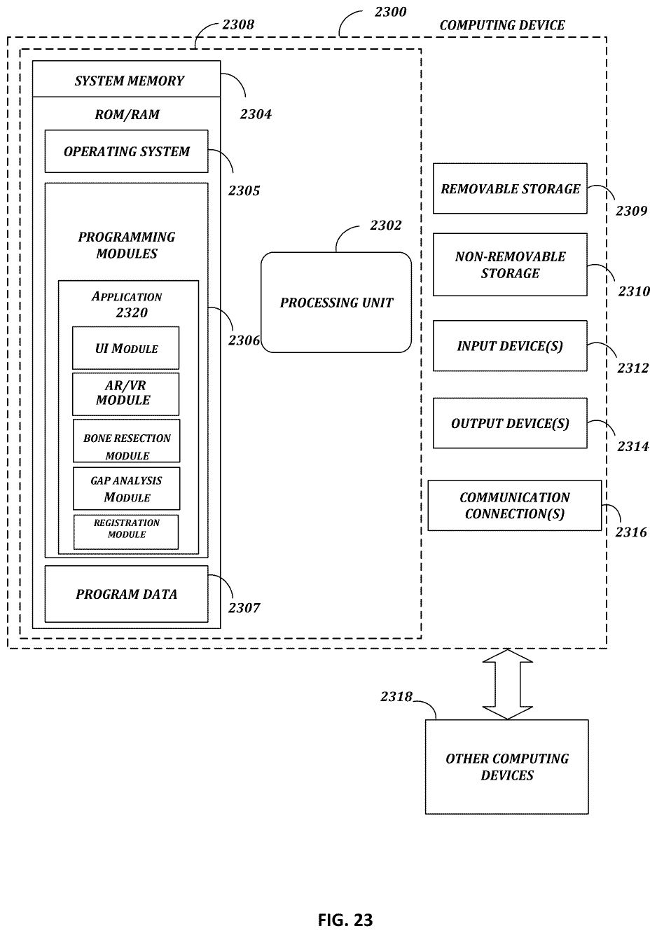

FIG. 23 is a block diagram of a system including a computing device for performing the various functions and methods disclosed herein.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Although modules are disclosed with specific functionality, it should be understood that functionality may be shared between modules, with some functions split between modules, while other functions duplicated by the modules. Furthermore, the name of the module should not be construed as limiting upon the functionality of the module. Moreover, each stage in the claim language can be considered independently without the context of the other stages. Each stage may contain language defined in other portions of this specifications. Each stage disclosed for one module may be mixed with the operational stages of another module. Each stage can be claimed on its own and/or interchangeably with other stages of other modules. The following claims will detail the operation of each module, and inter-operation between modules.

Although the stages are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones claimed below. Moreover, various stages may be added or removed from the without altering or deterring from the fundamental scope of the depicted methods and systems disclosed herein.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), an optical fiber, and quantum computing elements. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods'stages may be modified in any manner, including by reordering stages and/or inserting or deleting stages, without departing from the disclosure.

All rights including copyrights in the code included herein are vested in and the property of the Applicant. The Applicant retains and reserves all rights in the code included herein, and grants permission to reproduce the material only in connection with reproduction of the granted patent and for no other purpose.

### System Architecture and Components

Referring to FIG. 1, a system (100) for augmented reality surgical navigation may comprise an augmented reality surgical interface (102) configured to be worn by a user during surgical procedures. The augmented reality surgical interface (102) may include a head-mounted display device with integrated cameras, sensors, and processing capabilities. The interface (102) may provide stereoscopic visualization with depth perception and spatial tracking of the user's head position and orientation relative to the surgical field.

The system (100) may include a bone resection visualization module (104) configured to generate real-time three-dimensional displays of resected areas and removed anatomy. The visualization module (104) may receive input from imaging sensors (106) positioned to monitor the surgical field and track changes in bone geometry during resection procedures. The imaging sensors (106) may comprise depth cameras, structured light projectors, laser scanners, or combinations thereof to capture high-resolution spatial data of the surgical site.

A gap balancing module (108) may be configured to calculate flexion and extension gaps in real-time during knee replacement procedures. The gap balancing module (108) may incorporate patient-specific anatomical data, ligament tension measurements, and joint kinematics to determine optimal resection parameters. The module (108) may interface with force sensors (110) positioned on surgical instruments or trial components to measure gap distances and ligament tension during joint manipulation. A processor (112) may be configured to integrate visualization data from the bone resection visualization module (104) and gap balancing calculations from the gap balancing module (108) for coordinated display via the augmented reality surgical interface (102). The processor (112) may execute algorithms for real-time rendering, spatial registration, and user interface management to provide seamless integration of virtual and physical elements in the surgeon's field of view.

### Bone Resection Visualization System

The bone resection visualization module (104) may generate volumetric representations of removed bone tissue with precise spatial positioning relative to remaining anatomical structures. The module (104) may maintain a dynamic three-dimensional model of the surgical site that updates continuously as resection procedures progress. The visualization may include color-coded regions indicating planned resection boundaries, completed resections, and remaining bone tissue to be removed.

The system (100) may provide enhanced visibility controls allowing surgeons to selectively display or hide various anatomical elements. A visibility menu interface may enable toggling between views of original anatomy, current bone state, removed tissue volumes, and planned resection guides. The visualization module (104) may support transparency adjustments, cross-sectional views, and measurement overlays to facilitate precise surgical planning and execution.

The bone resection visualization module (104) may incorporate real-time volume calculations to quantify the amount of bone tissue removed during procedures. The system may display numerical values for removed volume, remaining bone thickness, and deviation from planned resection parameters. These quantitative metrics may be overlaid directly onto the surgical field or presented in dedicated information panels within the augmented reality interface.

The visualization system may support multiple viewing modes including wireframe representations, solid surface rendering, and hybrid displays combining virtual and physical elements. The module (104) may automatically adjust rendering parameters based on lighting conditions, viewing angle, and user preferences to maintain optimal visibility throughout the surgical procedure.

### Gap Balancing and Biomechanical Analysis

The gap balancing module (108) may calculate flexion and extension gap measurements using integrated force sensors and spatial tracking systems. The module may continuously monitor joint positioning during trial reduction and provide real-time feedback on gap symmetry, ligament tension, and joint alignment. The system may incorporate patient-specific biomechanical parameters derived from preoperative imaging and intraoperative measurements. The gap balancing calculations may account for ligament properties including stiffness, length, and attachment points to predict joint behavior under various loading conditions. The module (108) may model the effects of different resection depths and angles on final gap measurements, enabling surgeons to evaluate multiple scenarios before making irreversible cuts. The system may provide predictive analysis showing how proposed resection modifications will affect gap balance and joint kinematics.

The system may include automated adjustment recommendations based on measured gap discrepancies and target parameters. The gap balancing module (108) may calculate optimal resection depths for femoral and tibial surfaces to achieve desired gap measurements in both flexion and extension positions. The recommendations may be displayed as visual overlays indicating specific areas requiring additional bone removal or alternative implant sizing options.

The gap balancing system may support both manual measurement techniques and automated sensor-based data collection. Force sensors (110) integrated into trial components or surgical instruments may provide continuous monitoring of gap distances and ligament tension throughout the range of motion. The system may correlate these measurements with spatial tracking data to generate comprehensive biomechanical profiles for each patient.

### Registration and Alignment Mechanisms

The system (100) may include robust registration mechanisms that may be used to align virtual anatomical models with the patient's physical anatomy during surgical procedures. Registration may be critical for accurate visualization, resection guidance, implant positioning, and gap balancing operations. The system may support multiple registration methods that may be used independently or in combination to achieve optimal alignment between virtual and physical elements.

The system may include finger registration capabilities that may enable users to quickly initialize alignment by touching or pointing to known anatomical landmarks. During finger registration, the user may use a tracked finger to indicate specific points on the patient's anatomy that correspond to predetermined landmarks on the virtual model. The system may record the three-dimensional positions of the user's fingertip when contacting these landmarks and may match them to corresponding points on the virtual anatomy. This method may provide a rapid initial registration that may be particularly useful for establishing basic alignment at the beginning of a procedure.

Finger registration may be implemented through spatial tracking of the user's hand position via sensors in the augmented reality surgical interface (102). The system may provide visual guidance through the head-mounted display to direct the user to specific anatomical landmarks in a predetermined sequence. As each landmark is touched, the system may capture the position and may incrementally refine the alignment of the virtual model. The finger registration process may be completed within seconds, allowing for efficient workflow initiation without disrupting surgical preparation.

For situations requiring higher precision, the system may incorporate stylus registration capabilities. This method may utilize a tracked stylus or surgical tool for more accurate landmark selection compared to finger-based registration. The stylus tip may provide a smaller contact area and more definitive position information, potentially improving registration accuracy. The system may record the stylus tip position when pointing to or contacting specific anatomical features and may use these positions to establish spatial correspondence with the virtual model.

The stylus registration method may be particularly valuable for selecting small or precise anatomical features that may be difficult to indicate accurately with a finger. The system may track specialized surgical instruments that may already be part of the standard workflow, minimizing the need for additional equipment. Stylus registration may be used alone for the entire registration process or may be employed to refine an initial alignment established through finger registration.

Visual detection registration may provide an automated approach that may analyze camera data from the augmented reality surgical interface (102) to identify anatomical surfaces, shapes, or markers. The system may utilize computer vision algorithms to detect natural anatomical features or artificial fiducial markers placed on the surgical field. The detected visual features may be matched to corresponding elements in the virtual model to establish spatial alignment.

The visual detection registration may operate continuously in the background, analyzing the video feed from the integrated cameras in the augmented reality surgical interface (102). This method may not require direct user interaction with specific points on the anatomy, potentially allowing for hands-free registration. The system may employ machine learning algorithms trained on diverse anatomical datasets to recognize features across different patients and surgical scenarios.

A key advantage of the system may be its support for dynamic registration, which may continuously update the alignment between virtual and physical elements throughout the procedure. Rather than relying on a single initial registration that remains static, the system may track changes in patient position, tissue deformation, or surface anatomy in real-time. The virtual objects may be automatically adjusted to maintain proper alignment as the surgical field evolves during the procedure.

Dynamic registration may be particularly valuable during joint replacement procedures where the relative positions of anatomical structures may change as the joint is manipulated through different ranges of motion. The system may employ a combination of optical tracking, inertial measurement, and surface matching algorithms to maintain spatial coherence between virtual and physical elements. This continuous registration may help prevent misalignment that could otherwise occur due to patient movement or repositioning during surgery.

The system may be designed to leverage multiple registration methods in complementary ways to achieve optimal results. For example, a typical workflow may begin with manual finger registration to establish initial alignment, followed by more precise stylus registration of key landmarks. Once this baseline registration is established, visual and dynamic registration methods may maintain and refine the alignment over time without requiring additional user input.

The integration of multiple registration methods may provide redundancy and improved reliability, as each method may have different strengths and limitations. The system may intelligently weigh inputs from different registration sources based on confidence metrics and contextual factors. For example, visual detection may be given higher priority in well-illuminated areas with clear anatomical features, while inertial tracking data may be emphasized during rapid movements.

The system may provide feedback on registration quality and stability through visual indicators in the augmented reality display. These indicators may include alignment confidence scores, error estimates, or color-coded overlays showing areas of high and low registration accuracy. If registration quality degrades below acceptable thresholds, the system may alert the user and suggest re-registration or refinement of the current alignment.

Users may have the option to manually trigger re-registration at any point during the procedure if they observe misalignment between virtual and physical elements. The system may provide streamlined workflows for registration refinement that may focus on specific areas of concern without requiring a complete restart of the registration process. This adaptive approach may allow surgeons to maintain accurate alignment throughout lengthy procedures with minimal disruption to the surgical workflow.

The registration mechanisms may be tightly integrated with the bone resection visualization and gap balancing modules to ensure that all aspects of the system operate with consistent spatial references. Accurate registration may be fundamental to the system's ability to provide reliable guidance for resection boundaries, implant positioning, and biomechanical analysis. The multi-modal registration approach may contribute significantly to the overall precision and utility of the augmented reality surgical navigation system.

### Methods of Use and Surgical Workflow Integration

During preoperative planning, the system (100) may import patient-specific imaging data including CT scans, MRI images, and X-rays to generate three-dimensional anatomical models. The system may perform automated segmentation of bone structures, soft tissues, and anatomical landmarks to create baseline models for surgical planning. Surgeons may use the augmented reality interface to review patient anatomy, plan resection boundaries, and simulate surgical procedures before entering the operating room.

Intraoperatively, the system may provide continuous spatial registration between preoperative models and the actual surgical field. The augmented reality surgical interface (102) may overlay planned resection guides, anatomical landmarks, and measurement tools directly onto the patient's anatomy. The system may track surgical instrument positions and provide real-time guidance for bone cuts, implant positioning, and soft tissue management.

The bone resection visualization module (104) may update displays in real-time as surgeons perform bone cuts, showing the progression of resection relative to planned boundaries. The system may provide audio and visual alerts when resection approaches critical anatomical structures or deviates from planned parameters. Surgeons may use gesture controls or voice commands to adjust visualization settings, measurement tools, and display preferences without interrupting the surgical workflow.

The gap balancing module (108) may guide surgeons through systematic evaluation of joint gaps at multiple flexion angles. The system may provide step-by-step protocols for gap measurement, ligament balancing, and implant sizing decisions. Real-time feedback may indicate when target gap measurements are achieved or when additional adjustments are required to optimize joint function.

### Technical Advantages and Performance Benefits

The integrated visualization and gap balancing system may provide enhanced surgical precision compared to conventional manual techniques. Real-time feedback on resection progress and gap measurements may reduce the likelihood of over-resection or under-resection, leading to improved implant fit and joint function. The system may enable more consistent surgical outcomes by providing standardized measurement protocols and objective assessment criteria.

The augmented reality interface may reduce cognitive load on surgeons by consolidating multiple information sources into a unified display. Rather than dividing attention between separate monitors, measurement devices, and the surgical field, surgeons may access all relevant information through the head-mounted display. This integration may improve surgical efficiency and reduce the risk of errors associated with information transfer between systems.

The bone resection visualization capabilities may enhance surgical education and training by providing detailed documentation of resection geometry and technique variations. The system may record comprehensive data on surgical procedures for post-operative analysis, quality assurance, and continuous improvement initiatives. This data may support evidence-based refinement of surgical techniques and system algorithms.

The gap balancing automation may reduce surgical time by providing rapid calculations and adjustment recommendations. The system may eliminate manual measurement steps and reduce the number of trial reductions required to achieve optimal gap balance. Automated analysis may also identify subtle biomechanical issues that might be missed during manual assessment, leading to improved long-term patient outcomes.

### Alternative Embodiments and System Variations

In alternative embodiments, the system may be configured for use in various orthopedic procedures beyond knee replacement, including hip replacement, shoulder arthroplasty, and spinal fusion procedures. The bone resection visualization module (104) may be adapted to display procedure-specific anatomical structures and resection patterns appropriate for different surgical applications. The gap balancing algorithms may be modified to account for the biomechanical requirements of different joint systems.

The augmented reality surgical interface (102) may comprise various display technologies including optical see-through displays, video see-through systems, or hybrid configurations combining multiple display modalities. Alternative embodiments may include projection-based systems that overlay information directly onto the surgical field without requiring head-mounted displays. The interface may support multiple users simultaneously, enabling collaborative surgical procedures with shared visualization capabilities.

The imaging sensors (106) may comprise various sensing technologies including time-of-flight cameras, stereo vision systems, ultrasound transducers, or electromagnetic tracking devices. Alternative sensor configurations may provide different levels of accuracy, resolution, and real-time performance depending on specific surgical requirements. The system may support sensor fusion techniques combining multiple sensing modalities to enhance measurement accuracy and reliability.

The gap balancing module (108) may incorporate machine learning algorithms trained on large datasets of surgical outcomes to improve prediction accuracy and recommendation quality. Alternative embodiments may include adaptive algorithms that learn from individual surgeon preferences and technique variations to provide personalized guidance. The system may support integration with robotic surgical platforms to enable automated execution of recommended adjustments.

In some embodiments, the system may include wireless connectivity capabilities for remote consultation, surgical mentoring, and real-time collaboration with specialists at distant locations. The system may support cloud-based data storage and analysis services for comprehensive surgical documentation and outcome tracking. Alternative configurations may include portable or mobile implementations suitable for use in various clinical environments and resource-limited settings.

Referring to FIG. 1, the system architecture (100) illustrates the interconnected components of the augmented reality surgical assistance system. The central processing unit (112) coordinates data flow between the augmented reality surgical interface (102), bone resection visualization module (104), gap balancing module (108), and imaging sensors (106). The architecture may include network connectivity (114) for data synchronization with hospital information systems and cloud-based analytics platforms. The system may incorporate local data storage (116) for real-time processing and backup storage (118) for comprehensive surgical documentation. The medical imaging translation module (195) may be configured to convert medical imaging data into interactive three-dimensional models for use within the augmented reality surgical interface (102). The module (195) may process various imaging formats including CT scans, MRI images, X-rays, and ultrasound data to generate patient-specific anatomical representations. These representations may be dynamically updated during surgical procedures to reflect changes in bone geometry and tissue structure.

A user (115) may interact with the system (100) through various user controls (165) including desktop devices, handheld devices, and wearable devices. The user controls (165) may enable manipulation of virtual models, adjustment of visualization parameters, and selection of surgical planning options. The user controls (165) may further incorporate gesture recognition, voice commands, and touch interfaces to facilitate intuitive interaction with the augmented reality environment.

The AR/VR head mounted wearable device (125) may provide stereoscopic visualization with six degrees of freedom tracking to maintain accurate spatial registration between virtual content and the physical surgical field. The device (125) may include integrated cameras for passthrough visualization, depth sensors for surface mapping, and inertial measurement units for head position tracking. The wearable device (125) may be ergonomically designed to be comfortable during extended surgical procedures while maintaining optimal optical alignment.

The medical imaging translation module (195) may incorporate machine learning algorithms trained on large datasets of anatomical images to improve segmentation accuracy and feature recognition. The module (195) may automatically identify anatomical landmarks, calculate reference axes, and detect abnormalities to assist in surgical planning. The translation process may preserve spatial relationships and dimensional accuracy while optimizing rendering performance for real-time visualization.

The system (100) may enable the user (115) to toggle between different visualization modes using the user controls (165). These modes may include wireframe representations, solid surface rendering, cross-sectional views, and transparency adjustments. The user (115) may selectively display or hide specific anatomical structures, implant components, and surgical references to focus on relevant aspects of the procedure.

The AR/VR head mounted wearable device (125) may support multi-user collaboration by synchronizing spatial data across multiple devices. This may allow surgical teams to share a common view of the augmented reality environment while maintaining individual control over perspective and visualization preferences. The device (125) may include audio communication capabilities to facilitate team coordination during complex procedures.

The medical imaging translation module (195) may generate real-time volumetric representations of resected bone tissue with precise spatial positioning relative to remaining anatomical structures. The module (195) may calculate the volume of removed bone and provide quantitative feedback on resection depth, angles, and coverage. This information may be displayed directly in the user's field of view through the AR/VR head mounted wearable device (125).

The user (115) may utilize the user controls (165) to manipulate virtual implant components in relation to patient-specific anatomy. The system (100) may provide real-time feedback on implant positioning, alignment, and fit based on biomechanical parameters and surgical planning criteria. The user controls (165) may enable fine adjustments to implant position and orientation to optimize surgical outcomes.

The AR/VR head mounted wearable device (125) may incorporate eye tracking technology to enhance user interaction and reduce cognitive load during surgical procedures. The device (125) may automatically adjust visualization focus based on the user's gaze direction and may highlight relevant information in the user's current field of attention. This may improve workflow efficiency and reduce the need for manual interface navigation.

The medical imaging translation module (195) may support dynamic registration between preoperative imaging data and intraoperative reality. The module (195) may continuously update the spatial alignment between virtual models and physical anatomy based on real-time tracking data from the AR/VR head mounted wearable device (125). This may ensure accurate visualization despite patient movement or tissue deformation during the procedure.

The system (100) may provide comprehensive documentation capabilities by recording the surgical procedure from the perspective of the AR/VR head mounted wearable device (125). The recorded data may include spatial tracking information, user interactions, and system measurements to facilitate post-operative analysis and quality improvement. The documentation may be automatically associated with patient records through secure integration with hospital information systems.

In educational applications, the system (100) may enable experienced surgeons to guide trainees through procedures using shared augmented reality visualization. The AR/VR head mounted wearable device (125) may display guidance markers, optimal tool trajectories, and instructional annotations overlaid on the surgical field. The user controls (165) may allow instructors to highlight specific anatomical features or demonstrate proper technique in a spatially registered context.

Referring to FIGS. 2A and 2B, the bone alignment and resection planning flow (200) and implant sizing, fit, and positioning flow (250) may demonstrate the systematic approach to surgical planning and execution. The bone alignment flow (200) may include preoperative imaging analysis (202), anatomical landmark identification (204), resection boundary planning (206), and validation against patient-specific parameters (208). The implant sizing flow (250) may incorporate trial component evaluation (252), gap measurement protocols (254), biomechanical analysis (256), and final implant selection criteria (258).

As shown in FIGS. 2C, 2D, 2E, and 2F, the system may provide a comprehensive user interface for surgical navigation. FIG. 2C may display notification capabilities for case readiness, battery life, internet connectivity, and casting connectivity. FIG. 2D may illustrate the Instructions For Use (IFU) interface that may be required during first-time initialization to ensure proper system operation. FIG. 2E may present the Case Search Bar functionality, which may enable users to search for patient cases by number, last name, age, or gender. FIG. 2F may depict the Home Menu function, which may allow users to return to the main menu from any point in the workflow.

Referring to FIG. 3A, the patient scheduling interface (302) may include a digital calendar system that may allow surgical teams to schedule procedures based on surgeon availability, operating room resources, and patient priority. The interface may display color-coded status indicators for case readiness, including imaging data availability, preoperative planning completion, and equipment requirements.

FIG. 3B may illustrate the preoperative data preparation workflow (304), which may involve importing patient-specific imaging data from various modalities such as CT, MRI, and X-rays. The system may automatically segment anatomical structures and may generate three-dimensional models for use during surgery. Surgeons may review and approve these models through the AR interface prior to the scheduled procedure.

FIG. 3C may depict the intraoperative data capture process (306), wherein the AR system may continuously record surgical field imagery, instrument tracking data, and procedural metrics throughout the operation. The system may timestamp all captured data and may associate it with the specific patient case for comprehensive documentation.

FIG. 3D may show the postoperative documentation workflow (308), which may include automated generation of surgical reports based on intraoperative data. The system may compile key metrics such as procedure duration, implant positioning measurements, and bone resection volumes into structured reports that may be reviewed and annotated by the surgical team.

FIG. 3E may illustrate the data synchronization process (310) that may enable bidirectional information exchange between the AR surgical system and hospital information systems. Patient demographic data, surgical scheduling information, and procedural outcomes may be automatically transferred to electronic health records and surgical databases, while maintaining HIPAA compliance and data security protocols.

Referring to FIGS. 4A and 4B, the automated processing and surgical planning flow may demonstrate the system's capability for intelligent analysis and recommendation generation. The flow may include automated image segmentation (402), anatomical model generation (404), surgical parameter optimization (406), and risk assessment protocols. The system may incorporate machine learning algorithms for continuous improvement of planning accuracy and outcome prediction.

As shown in FIG. 4A, the automated processing flow may include a data acquisition phase where patient-specific imaging data may be collected through various modalities including CT scans, MRI, and radiographs. These imaging data may be processed through a specialized segmentation pipeline that may identify and isolate relevant anatomical structures with high precision. The segmentation algorithms may utilize deep learning networks trained on extensive datasets to accurately delineate bone boundaries, soft tissue interfaces, and critical anatomical landmarks.

FIG. 4B may illustrate the model generation phase where the segmented data may be transformed into interactive three-dimensional anatomical models. These models may be rendered with variable opacity settings to enable visualization of internal structures when needed. The system may automatically identify key anatomical landmarks and may establish reference axes for subsequent surgical planning. The anatomical models may be optimized for real-time rendering within the augmented reality environment while maintaining anatomical accuracy.

FIG. 4C may depict the surgical planning optimization workflow where the system may analyze the patient-specific anatomical models to generate recommended resection planes, implant positions, and alignment parameters. The optimization algorithms may consider multiple factors including mechanical alignment, kinematic alignment, soft tissue balancing, and implant-specific requirements. The system may simulate post-operative biomechanics and may predict functional outcomes based on different surgical approaches, enabling the surgeon to evaluate various scenarios before making final decisions.

Referring to FIGS. 5A and 5B, the loading process in Unity App for surgery flow (500) illustrates the software initialization and calibration procedures. The flow may include application startup (502), hardware connectivity verification (504), spatial calibration protocols (506), and user interface initialization (508). The system may perform automated system checks (510) and provide status feedback to ensure optimal performance before surgical procedures begin.

Referring to FIG. 5A, the loading process may begin with a user login sequence where the user may select their profile through the user selection menu. The user may be required to authenticate by touching or selecting the user icon located at the bottom left of the interface, adjacent to the time display. This authentication step may ensure that user-specific settings and preferences are loaded for the surgical session.

Referring to FIG. 5B, following successful authentication, the main menu interface may be displayed, presenting the user with multiple function options arranged in a grid layout. These options may include Translation, Anchor, Cast, 3D Visibility, Resections, Implant System, and Registration. Each function may be represented by a distinctive icon and label to facilitate intuitive navigation. The system may display status indicators along the bottom edge of the interface, including connectivity status, battery life, and the current time, allowing the user to monitor system readiness throughout the initialization process.

Referring to FIGS. 6A and 6B, the intraoperative AR guidance process flow (600) may demonstrate real-time surgical assistance capabilities. The flow may include continuous spatial tracking (602), real-time visualization updates (604), measurement and analysis functions (606), and adaptive guidance adjustments (608). The system may provide multi-modal feedback including visual overlays (610), audio alerts (612), and haptic guidance (614) to support surgical decision-making. The system may further enable precise control of virtual objects through a translation interface as shown in FIG. 6B, which may include incremental adjustments along X, Y, and Z axes as well as rotational controls for flexion and extension. The translation interface may additionally provide specialized alignment controls for anatomical references such as femoral and tibial positioning. Referring to FIGS. 6C, 6D, and 6E, the bone resection visualization module may provide detailed visualization of femoral and tibial alignment axes and adjustment capabilities. As shown in FIG. 6C, after selecting the Femoral option in FIG. 6B, the system may display three primary femoral alignment axes: an anteroposterior axis (AP) (606) extending vertically through the center of the femur, a transepicondylar axis (TEA) (608) extending horizontally across the femoral condyles, and a posterior condylar axis (PCA) (610) running horizontally along the posterior aspects of the femoral condyles. The user may interact with these axes through hand gestures, allowing precise adjustment of femoral component rotation and alignment during surgical planning.

FIG. 6D may illustrate tibial rotation adjustment capabilities after selection from the options in FIG. 6B. The system may display a top-down view of the tibial component with medial (614) and lateral (612) rotation controls. The user may manipulate the tibial component through hand gestures to adjust rotational alignment, with the medial side labeled “med” and the lateral side labeled “lat” for clear orientation. This visualization may enable surgeons to optimize tibial component rotation based on patient-specific anatomy and biomechanical considerations.

FIG. 6E may demonstrate the adjustable mechanical axis functionality for both femur and tibia, providing visualization for varus/valgus and flexion/extension adjustments. The system may display side-by-side radiographic-style depictions of the knee joint with adjustable mechanical axis indicators (616, 618, 620, 622). The user may manipulate instruments at the femoral end to modify the mechanical axis alignment, with visual feedback showing how these adjustments affect overall limb alignment. These visualization tools may allow surgeons to evaluate different alignment scenarios and their potential impact on joint biomechanics before making actual bone resections.

Referring to FIGS. 7A and 7B, the postoperative documentation process flow may include automated report generation (702), outcome metric calculation (704), comparative analysis with preoperative plans (706), and integration with quality assurance protocols (708). The system may support customizable reporting formats (710) and automated data export to research databases (712).

Referring to FIGS. 8 and 9A, 9B, the enhanced surgical case scheduling and automated processing flows (800, 900) may demonstrate advanced workflow integration capabilities. The scheduling flow (800) may include intelligent case prioritization (802), resource allocation optimization (804), and predictive scheduling based on case complexity (806). The automated processing flow (900) may incorporate advanced imaging analysis (902), multi-modal data fusion (904), and predictive modeling for surgical outcomes (906).

Referring to FIGS. 10A and 10B, the augmented reality surgical guidance flow (1000) may illustrate the comprehensive intraoperative assistance system. The flow may include real-time anatomy registration (1002), dynamic visualization updates (1004), interactive measurement tools (1006), and adaptive guidance based on surgical progress (1008). The system may provide contextual information displays (1010) and intelligent alert systems (1012) to enhance surgical awareness and decision-making.

FIG. 10C may show a three-dimensional visualization of knee joint resection planes with the following features. In one or more embodiments, the figure may display a three-dimensional rendering of a knee joint with multiple resection planes visualized in an augmented reality environment. Two semi-transparent hands may be shown interacting with the virtual model, demonstrating the user manipulation capabilities of the system. The distal femur may be displayed with several labeled cut planes including the anterior femur cut (1005), distal femur cut (1010), posterior femur cut (1015), anterior chamfer cut (1020), and posterior chamfer cut (1025). The proximal tibia may be shown with a horizontal cut line (1030) representing the tibial resection plane. The visualization may enable surgeons to interact with and manipulate these resection planes in real-time, allowing for precise planning and adjustment of bone cuts during knee arthroplasty procedures. The three-dimensional nature of the display may provide enhanced spatial understanding of the relationship between different resection planes and their impact on the overall alignment and balance of the knee joint. The interactive nature of the visualization may allow surgeons to evaluate multiple resection scenarios before making actual bone cuts, potentially improving surgical outcomes through more precise preoperative planning.

Referring to FIGS. 11A and 11B, a schematic diagram may illustrate an intraoperative guidance process flow for the augmented reality surgical navigation system. FIG. 11A may depict a knee joint visualization with bone resection guidance overlays. The visualization may include a three-dimensional rendering of a distal femur and proximal tibia with planned resection planes highlighted in contrasting colors. The femoral component may be shown with anterior, posterior, distal, and chamfer cuts clearly delineated through semi-transparent overlays. The tibial component may display a proximal cut line with adjustable positioning indicators.

FIG. 11B may illustrate the gap balancing visualization interface that may be presented to the surgeon during the procedure. The interface may display real-time measurements of flexion and extension gaps between the femoral and tibial components. Numerical values indicating gap distances in millimeters may be shown for both medial and lateral compartments. The visualization may include color-coded indicators that may signal whether measured gaps fall within target parameters, with green indicating optimal range, yellow suggesting borderline measurements, and red highlighting significant deviations requiring adjustment.

The gap balancing interface may further provide graphical representations of ligament tension through vector arrows or tension meters positioned along the medial and lateral aspects of the joint. These indicators may dynamically update as the surgeon manipulates the joint through various degrees of flexion and extension. The system may display predictive modeling of how proposed resection adjustments may affect gap measurements, enabling the surgeon to evaluate multiple scenarios before making final cuts.

Referring to FIGS. 12A, 12B, 12C, 12D, 12E, 12F, 12G, the intraoperative guidance and postoperative documentation process flows may include multiple phases of surgical support. Referring to FIG. 12A, a graphical user interface (1200A) may be displayed via the augmented reality surgical interface. The interface may present multiple function options including a translator function, an anchor function, a cast function, a 3D visibility function, a resections function, an implant system function, and a registration function. These functions may be arranged as selectable buttons on the interface, enabling the surgeon to access different capabilities during the intraoperative phase. The interface may display status information including time and system status along a bottom portion, along with branding information displayed centrally.

Referring to FIG. 12B, the interface (1200B) may present multiple interaction modality options for surgical guidance. These options may include an index finger selection (1202) enabling direct finger-based interaction with virtual elements, a stylus selection (1204) supporting precision input using a dedicated instrument, a visual detection selection (1206) enabling camera-based recognition of surgical elements, and a return option (1208) for navigating back to previous interface screens. The system may adapt its interaction methods based on the selected modality to accommodate different surgical scenarios and user preferences.

Referring to FIG. 12C, after selecting the index finger registration option (1202), the system may initiate a finger index registration process (1205) where the user's finger may interact with a registration pattern applied to the surgical site. The pattern may comprise a grid of small dots or markers applied to the patient's anatomy that may enable precise spatial tracking and registration of the augmented reality elements relative to the physical surgical field.

Referring to FIG. 12D, following successful finger registration, the system may generate an image projection (1210) overlaying virtual surgical guidance elements onto the patient's anatomy. The projection may include three-dimensional representations of implant components, resection planes, or anatomical landmarks that may be spatially anchored to the physical anatomy through the registration process. The surgeon may continue to use finger-based interaction to manipulate these virtual elements during the procedure.

Referring to FIG. 12E, when the stylus option (1204) is selected, the system may enable stylus or surgical tool registration and image projection (1215). This modality may allow the surgeon to use a dedicated stylus or an actual surgical instrument as a precise pointing and interaction device. The system may track the position and orientation of the stylus relative to the patient's anatomy and may use this information to enable accurate manipulation of virtual surgical planning elements.

Referring to FIG. 12F, after selection in FIG. 12B, the system may implement dynamic visual registration and object recognition (1220). This functionality may enable the system to automatically identify anatomical structures, surgical instruments, or reference markers within the surgical field. The system may display visual feedback indicating successful recognition, such as highlighting recognized objects with bounding boxes or overlaying identification labels directly in the surgeon's field of view.

Referring to FIG. 12G, following dynamic visual registration and object recognition, the system may generate image projections (1225) that may be precisely aligned with the recognized objects. These projections may include surgical planning information, such as resection planes, implant positioning guides, or measurement references, overlaid directly onto the corresponding anatomical structures. The projections may update in real-time as the surgical field changes, maintaining accurate spatial registration throughout the procedure.

The intraoperative guidance process flow may enable continuous monitoring of surgical progress through spatial tracking and real-time visualization, providing quantitative metrics on resection accuracy and gap measurements. The system may adaptively adjust recommendations based on intraoperative findings, supporting surgical decision-making with contextually relevant information. The postoperative documentation flow may incorporate comprehensive data analysis of the completed procedure, correlating preoperative plans with actual surgical results, and generating feedback that may inform future surgical planning and execution.

Referring to FIGS. 13A and 13B, the graphical user interface (1300) implemented in the head-mounted display may present a slicer tool box image (1302), slicer tool plane (1304), femur 3D model rendering (1306), and tibia 3D model rendering (1308). The interface may provide intuitive controls for manipulating anatomical models, adjusting visualization parameters, and accessing measurement tools. The slicer tool functionality may enable cross-sectional analysis of bone structures and precise visualization of resection planes.

Referring to FIGS. 14A, 14B, 14C, 14D, and 14E, the visibility menu interface may include options for toggling anatomical structures, adjusting transparency levels, and controlling overlay visibility. The interface may support customizable display presets and user-specific preference settings to optimize visualization for different surgical phases and user requirements. Each figure demonstrates different aspects of the selective display control capability, with FIG. 14A showing the anterior femur cut visualization, FIG. 14B displaying the distal femur cut, FIG. 14C illustrating the posterior femur cut, and FIGS. 14D and 14E presenting different chamfer cut visualizations. These visualization options may enable the surgeon to selectively view specific anatomical structures and planned resections while maintaining spatial relationships between virtual objects and physical anatomy.

Referring to FIG. 15, the anchoring menu interface (1500) may illustrate the virtual locking system configured to lock images and three-dimensional renderings in space. The system may provide spatial anchoring controls for the proximal cut line of the system for bone resection. The proximal cut line may be positioned across the top of the tibia, extending from the medial to lateral edge as shown in FIG. 15. The anchoring system may enable surgeons to maintain the proximal cut line in a fixed spatial position during the procedure, preventing inadvertent displacement of this critical resection reference. The vertical double-headed arrow at the right end of the proximal cut line may indicate the adjustable height or depth of the resection, which may be locked in place once the optimal position is determined.

Referring to FIGS. 16A and 16B may show the various icons representing options available from the main menu for surgeons using the platform. FIG. 16A may display a collection of functional icons organized in a grid layout, including three-dimensional visualization tools, targeting aids, cross-sectional imaging controls, bone selection options, fracture assessment tools, cutting instruments, imaging device interfaces, joint visualization options, casting capabilities, individual bone selection, anatomical cross-section views, and anchoring functions. FIG. 16B may illustrate directional control icons for precise manipulation of virtual objects, including positive and negative movement along X, Y, and Z axes, rotation controls in multiple directions, information access, search functionality, home navigation, settings configuration, and system reset options.

### Method Flowcharts for Augmented Reality Surgical Assistance System

Referring to FIG. 17, a method flowchart (1700) may illustrate the enhanced anchoring process for three-dimensional visualized models in an augmented reality surgical environment. The method may begin with an initialization step (1702) where the system may establish a spatial reference frame and load patient-specific anatomical models. The system may then proceed to a multi-object anchoring step (1704) where multiple virtual objects including anatomical structures and surgical instruments may be independently selected and anchored within the augmented reality environment. The method may continue with a hierarchical locking structure implementation step (1706) where parent-child relationships between anchored objects may be established to maintain relative positioning during manipulation. The system may then perform a dynamic anchor adjustment step (1708) where anchored objects may automatically adapt to changes in the surgical field while maintaining their defined spatial relationships. The method may include an anchor verification step (1710) where the system may validate the stability and accuracy of anchored objects through visual and computational means. The method may conclude with an anchor release step (1712) where selected anchors may be selectively disengaged to allow for repositioning or removal of virtual objects as needed during the surgical procedure.

Referring to FIG. 18, a method flowchart (1800) may demonstrate the hand interaction process for manipulating virtual objects in the augmented reality surgical environment. The method may begin with a gesture recognition initialization step (1802) where the system may calibrate and activate hand tracking sensors to detect user hand positions and movements. The method may proceed to a close grab function step (1804) where the system may enable direct manipulation of virtual objects when the user's hand is within a predetermined proximity threshold to the target object. The method may continue with a distance grab function step (1806) where users may select and manipulate objects from a greater distance using pointing gestures and pinch movements. The system may then implement a CT plane viewer interaction step (1808) where specific hand gestures may control the positioning and orientation of cross-sectional planes through volumetric medical imaging data. The method may include a haptic feedback generation step (1810) where the system may provide tactile sensations to the user through controllers or wearable devices to enhance precision during object manipulation. The method may conclude with a visual confirmation step (1812) where the system may display visual indicators confirming successful object selection, movement, or placement to provide user feedback during interaction.

Referring to FIG. 19, a method flowchart (1900) may illustrate the measurement process for analyzing predetermined areas of interest in the augmented reality surgical environment. The method may begin with a measurement initialization step (1902) where the system may activate measurement tools and establish reference scales based on patient-specific anatomical data. The method may proceed to an automated measurement tool deployment step (1904) where the system may automatically identify key anatomical landmarks and generate relevant measurement references. The method may continue with a custom measurement protocol implementation step (1906) where surgeons may define specific measurement sequences tailored to particular surgical procedures or patient conditions. The system may then perform a real-time calculation step (1908) where measurements may be continuously updated as virtual or physical objects are manipulated within the surgical field. The method may include a measurement visualization step (1910) where calculated values may be displayed directly within the augmented reality environment alongside the measured structures. The method may conclude with a measurement data recording step (1912) where all measurements may be logged and stored for documentation and post-operative analysis.

Referring to FIG. 20, a method flowchart (2000) may provide additional detail on the enhanced hand interaction process for the augmented reality surgical assistance system. The method may begin with a hand tracking initialization step (2002) where the system may detect and begin monitoring the user's hands within the field of view. The method may proceed to a close grab execution step (2004) where direct manipulation of virtual objects may occur when hands are in close proximity to the target. The method may continue with a distance grab execution step (2006) where ray-casting and pointing gestures may enable selection and manipulation of distant objects. The system may then implement an advanced gesture recognition step (2008) where complex hand movements and finger positions may be interpreted as specific commands or actions. The method may include a multi-hand tracking coordination step (2010) where simultaneous tracking of both hands may enable bimanual interactions such as scaling, rotating, or performing complementary actions. The method may conclude with an adaptive interaction zone adjustment step (2012) where the system may dynamically modify interaction parameters based on user preferences, surgical context, and environmental conditions.

Referring to FIG. 21, a system architecture flowchart (2100) may illustrate the data flow within the augmented reality surgical assistance system. The process may begin with a data acquisition step (2102) where patient imaging, surgical planning information, and real-time sensor data may be collected and prepared for processing. The method may proceed to a data preprocessing step (2104) where raw input data may be filtered, aligned, and optimized for use within the system. The method may continue with a processing module execution step (2106) where specialized algorithms may transform the preprocessed data into actionable information and visualizations. The system may then implement a storage management step (2108) where processed data, user preferences, and system states may be organized and maintained for efficient retrieval. The method may include a communication interface operation step (2110) where data may be exchanged between system components, external devices, and network resources to enable collaborative functionality. The method may conclude with a modular expansion integration step (2112) where additional capabilities may be incorporated into the existing system architecture through standardized interfaces and protocols.

Referring to FIG. 22, a method flowchart (2200) may demonstrate the systematic approach to providing augmented reality surgical assistance. The method may begin with a system initialization step (2202) where hardware components may be activated, software modules may be loaded, and initial system checks may be performed. The method may proceed to a calibration protocol execution step (2204) where the system may align virtual and physical spaces, adjust for user-specific parameters, and verify tracking accuracy. The method may continue with a real-time processing step (2206) where continuous data streams from cameras, sensors, and user inputs may be processed to update the augmented reality environment. The system may then implement a validation procedure step (2208) where the accuracy of virtual object positioning, measurements, and system responses may be verified against established benchmarks. The method may include an error handling protocol execution step (2210) where the system may detect, classify, and respond to various error conditions while maintaining essential functionality. The method may conclude with an adaptive algorithm adjustment step (2212) where the system may modify its behavior based on observed patterns, user feedback, and changing surgical conditions to optimize performance throughout the procedure.

FIG. 23 is a block diagram of a system including computing device 2300. Consistent with an embodiment of the disclosure, the aforementioned memory storage and processing unit may be implemented in a computing device, such as computing device 2300 of FIG. 23. Any suitable combination of hardware, software, or firmware may be used to implement the memory storage and processing unit. For example, the memory storage and processing unit may be implemented with computing device 2300 or any of other computing devices 2318, in combination with computing device 2300. The aforementioned system, device, and processors are examples and other systems, devices, and processors may comprise the aforementioned memory storage and processing unit, consistent with embodiments of the disclosure.

With reference to FIG. 23, a system consistent with an embodiment of the disclosure may include a computing device, such as computing device 2300. In a basic configuration, computing device 2300 may include at least one processing unit 2302 and a system memory 2304. Depending on the configuration and type of computing device, system memory 2304 may comprise, but is not limited to, volatile (e.g. random access memory (RAM)), non-volatile (e.g. read-only memory (ROM)), flash memory, or any combination. System memory 2304 may include operating system 2305, one or more programming modules 2306, and may include a program data 2307. Operating system 2305, for example, may be suitable for controlling computing device 2300's operation. In one embodiment, programming modules 2306 may include a user interface module, a AR/VR module, a bone resection module, a gap analysis module, a registration module, and application 2320. Furthermore, embodiments of the disclosure may be practiced in conjunction with a graphics library, other operating systems, or any other application program and is not limited to any particular application or system. This basic configuration is illustrated in FIG. 23 by those components within a dashed line 2308.

Computing device 2300 may have additional features or functionality. For example, computing device 2300 may also include additional data storage devices (removable and/or non-removable) such as, for example, magnetic disks, optical disks, or tape. Such additional storage is illustrated in FIG. 23 by a removable storage 2309 and a non-removable storage 2310. Computer storage media may include volatile and nonvolatile, removable and non-removable media implemented in any method or technology for storage of information, such as computer readable instructions, data structures, program modules, or other data. System memory 2304, removable storage 2309, and non-removable storage 2310 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 2300. Any such computer storage media may be part of device 2300. Computing device 2300 may also have input device(s) 2312 such as a keyboard, a mouse, a pen, a sound input device, a touch input device, etc. Output device(s) 2314 such as a display, speakers, a printer, etc. may also be included. The aforementioned devices are examples and others may be used.

Computing device 2300 may also contain a communication connection 2316 that may allow device 2300 to communicate with other computing devices 2318, such as over a network in a distributed computing environment, for example, an intranet or the Internet. Communication connection 2316 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 2304, including operating system 2305. While executing on processing unit 2302, programming modules 2306 (e. g a user interface module, a AR/VR module, a bone resection module, a gap analysis module, a registration module, and application 2320) may perform processes including, for example, one or more of method 1700, 1800, 1900, 2000, 2100, or 2200's stages as described herein. The aforementioned process is an example, and processing unit 2302 may perform other processes. Other programming modules that may be used in accordance with embodiments of the present disclosure may include electronic mail and contacts applications, word processing applications, spreadsheet applications, database applications, slide presentation applications, drawing or computer-aided application programs, etc.

FIG. 17 shows a method of operating a virtual locking system for three-dimensional visualized models, the method may comprise:
receiving, via a graphical user interface (GUI) implemented in a display of a head mounted wearable device, a selection of an anchoring menu option;
displaying, via the GUI, an anchoring menu image comprising a plurality of selectable virtual object types;
receiving, via the GUI, a selection of at least one virtual object type from the plurality of selectable virtual object types;
enabling manipulation of a virtual object corresponding to the selected virtual object type through at least one of direct grabbing or translation controls;
receiving, via the GUI, an anchoring command to fix a spatial position of the virtual object; and
anchoring the virtual object in a defined spatial relationship relative to at least one of a physical environment, an anatomical reference, or a coordinate system, wherein the virtual object maintains positional and rotational stability despite user movement.

Regarding FIG. 18, it provides for a method of providing hand interaction functionality in an augmented reality surgical environment, the method may comprise:
displaying, via a graphical user interface (GUI) implemented in a display of a head mounted wearable device, a hand interaction image comprising a close grab function and a distance grab function;
displaying, via the GUI, a CT plane viewer image comprising at least one cross-sectional view of patient-specific anatomical data;
detecting a hand gesture corresponding to one of the close grab function or the distance grab function;
determining whether the detected hand gesture is within a predetermined proximity threshold of a virtual object;
when the detected hand gesture is within the predetermined proximity threshold, enabling direct manipulation of the virtual object through the close grab function; and
when the detected hand gesture exceeds the predetermined proximity threshold, enabling remote manipulation of the virtual object through the distance grab function.

Regarding FIG. 19, it teaches a method of providing measurement functionality in an augmented reality surgical environment, the method may comprise:
receiving, via a graphical user interface (GUI) implemented in a display of a head mounted wearable device, a selection of a measurement function;
displaying, via the GUI, a measurement image comprising one or more predetermined areas of interest on a virtual anatomical model;
receiving, via the GUI, a selection of at least one predetermined area of interest from the one or more predetermined areas of interest;
calculating one or more measurements associated with the selected predetermined area of interest;
displaying, via the GUI, the one or more measurements in spatial association with the selected predetermined area of interest; and
updating the one or more measurements in real-time in response to manipulation of the virtual anatomical model.

Regarding FIG. 20, it teaches a method of providing hand interaction functionality in an augmented reality surgical environment, the method may comprise:
displaying, via a graphical user interface (GUI) implemented in a display of a head mounted wearable device, a hand interaction image comprising a close grab function and a distance grab function;
detecting a hand gesture corresponding to one of the close grab function or the distance grab function;
determining a spatial relationship between the detected hand gesture and a virtual object;
when the spatial relationship indicates the hand gesture is within a first predetermined distance threshold, activating the close grab function to enable direct manipulation of the virtual object;
when the spatial relationship indicates the hand gesture is beyond the first predetermined distance threshold but within a second predetermined distance threshold, activating the distance grab function to enable remote manipulation of the virtual object; and
providing visual feedback via the GUI indicating which grab function is currently active.

Regarding FIG. 21, it teaches a system for augmented reality surgical navigation may comprise:
a head-mounted wearable device comprising a display, one or more cameras, and one or more sensors;
a processor in communication with the head-mounted wearable device;
a memory storing instructions that, when executed by the processor, may cause the system to:
display, via a graphical user interface (GUI) implemented in the display, a main menu comprising a plurality of selectable functions;
receive, via the GUI, a selection of at least one function from the plurality of selectable functions;
display, via the GUI, a function-specific interface corresponding to the selected function;
detect, via the one or more cameras and the one or more sensors, user input comprising at least one of a hand gesture, a head movement, or a voice command;
process the detected user input to determine a corresponding action within the function-specific interface; and
update the GUI to reflect the corresponding action.

Regarding FIG. 22, it provides for a system for augmented reality surgical navigation, further to the system of FIG. 21, wherein the memory may store additional instructions that, when executed by the processor, may cause the system to:
establish a spatial registration between a virtual anatomical model and a physical anatomy of a patient;
maintain the spatial registration during movement of at least one of the head-mounted wearable device or the patient;
enable selective visualization of different anatomical structures within the virtual anatomical model;
display virtual resection planes in spatial alignment with the virtual anatomical model;
enable adjustment of the virtual resection planes through at least one of direct manipulation or menu-based controls; and
provide real-time feedback on biomechanical parameters based on the position of the virtual resection planes relative to the virtual anatomical model.

### System Integration and Data Management

The system (100) may include a comprehensive data management platform configured to capture, store, and analyze surgical procedure data for quality assurance and outcome correlation. The platform may record detailed metrics including resection geometry, gap measurements, surgical timing, instrument trajectories, and deviations from planned parameters. This data may be automatically synchronized with electronic health records and surgical databases to support longitudinal outcome studies and evidence-based practice improvements.

The system may provide integration capabilities with existing hospital information systems, surgical scheduling platforms, and medical imaging networks. The integration may enable seamless data exchange between preoperative planning systems, intraoperative guidance platforms, and postoperative documentation tools. The system may support standard medical data formats including DICOM, HL7, and FHIR to ensure interoperability with diverse clinical environments.

### Performance Optimization and Calibration

The system may incorporate automated calibration procedures to maintain accuracy and reliability of measurements throughout surgical procedures. The calibration may include spatial registration verification, sensor alignment confirmation, and display accuracy validation. The system may perform continuous background calibration using reference markers, anatomical landmarks, or dedicated calibration targets positioned within the surgical field.

The processor (112) may implement adaptive algorithms that optimize system performance based on real-time conditions including lighting variations, user movement patterns, and surgical site characteristics. The optimization may include automatic adjustment of rendering parameters, sensor sensitivity, and tracking algorithms to maintain consistent performance across diverse surgical environments and user preferences.

The disclosed system for augmented reality surgical navigation with integrated bone resection visualization and gap balancing represents a comprehensive solution for enhancing surgical precision and outcomes in orthopedic procedures. The integration of real-time visualization capabilities with automated biomechanical analysis provides surgeons with unprecedented insight into surgical progress and joint mechanics. The system may enable more consistent, accurate, and efficient surgical procedures while supporting comprehensive documentation and continuous improvement initiatives. The modular architecture and alternative embodiments described herein provide flexibility for adaptation to various surgical specialties and clinical environments, ensuring broad applicability and scalability of the disclosed technology.

What is claimed is:

1. A system for augmented reality surgical navigation comprising:
an augmented reality surgical interface configured to be worn by a user and comprising a head-mounted display device with integrated cameras and sensors;
a bone resection visualization module configured to generate real-time three-dimensional displays of resected areas and removed anatomy during surgical procedures;
a gap balancing module configured to calculate flexion and extension gaps and provide automatic adjustment recommendations based on patient-specific biomechanical parameters;
imaging sensors positioned to monitor a surgical field and track changes in bone geometry during resection procedures; and
a processor configured to integrate visualization data from the bone resection visualization module and gap balancing calculations from the gap balancing module for coordinated display via the augmented reality surgical interface.

2. The system of claim 1, wherein the bone resection visualization module is configured to generate volumetric representations of removed bone tissue with precise spatial positioning relative to remaining anatomical structures.

3. The system of claim 1, wherein the gap balancing module incorporates force sensors configured to measure gap distances and ligament tension during joint manipulation.

4. The system of claim 1, wherein the imaging sensors comprise depth cameras, structured light projectors, laser scanners, or combinations thereof.

5. The system of claim 1, wherein the bone resection visualization module provides enhanced visibility controls allowing selective display of original anatomy, current bone state, removed tissue volumes, and planned resection guides.

6. The system of claim 1, wherein the gap balancing module is configured to calculate optimal resection depths for femoral and tibial surfaces to achieve target gap measurements in both flexion and extension positions.

7. The system of claim 1, wherein the processor is configured to provide real-time volume calculations quantifying bone tissue removed during procedures.

8. The system of claim 1, wherein the augmented reality surgical interface supports multiple viewing modes comprising wireframe representations, solid surface rendering, and hybrid displays combining virtual and physical elements.

9. The system of claim 1, wherein the gap balancing module is configured to model effects of different resection depths and angles on gap measurements and provide predictive analysis of proposed resection modifications.

10. The system of claim 1, further comprising a surgical training module configured to provide immersive educational experiences using the integrated visualization and gap balancing capabilities.

11. A method for augmented reality surgical navigation comprising:
displaying, via an augmented reality surgical interface worn by a user, real-time three-dimensional visualizations of a surgical field;
generating, via a bone resection visualization module, displays of resected areas and removed anatomy during surgical procedures;
calculating, via a gap balancing module, flexion and extension gaps based on patient-specific biomechanical parameters;
monitoring, via imaging sensors, the surgical field to track changes in bone geometry during resection procedures;
providing automatic adjustment recommendations based on the calculated gaps; and
integrating, via a processor, the visualization data and gap balancing calculations for coordinated display via the augmented reality surgical interface.

12. The method of claim 11, further comprising generating volumetric representations of removed bone tissue with spatial positioning relative to remaining anatomical structures.

13. The method of claim 11, further comprising measuring gap distances and ligament tension during joint manipulation using force sensors.

14. The method of claim 11, further comprising providing enhanced visibility controls for selective display of original anatomy, current bone state, removed tissue volumes, and planned resection guides.

15. The method of claim 11, further comprising calculating optimal resection depths for femoral and tibial surfaces to achieve target gap measurements in both flexion and extension positions.

16. The method of claim 11, further comprising providing real-time volume calculations quantifying bone tissue removed during procedures.

17. The method of claim 11, further comprising modeling effects of different resection depths and angles on gap measurements and providing predictive analysis of proposed resection modifications.

18. A non-transitory computer-readable medium storing instructions that, when executed by a processor, cause the processor to:
control an augmented reality surgical interface to display real-time three-dimensional visualizations of a surgical field;
generate, via a bone resection visualization module, displays of resected areas and removed anatomy during surgical procedures;
calculate, via a gap balancing module, flexion and extension gaps based on patient-specific biomechanical parameters;
process data from imaging sensors monitoring the surgical field to track changes in bone geometry during resection procedures;
provide automatic adjustment recommendations based on the calculated gaps; and
integrate the visualization data and gap balancing calculations for coordinated display via the augmented reality surgical interface.

19. The non-transitory computer-readable medium of claim 18, wherein the instructions further cause the processor to generate volumetric representations of removed bone tissue with spatial positioning relative to remaining anatomical structures.

20. The non-transitory computer-readable medium of claim 18, wherein the instructions further cause the processor to calculate optimal resection depths for femoral and tibial surfaces to achieve target gap measurements in both flexion and extension positions.
